SEQUENCE LISTING (264 sequences)

SEQ ID NO: 1 - RNA, 23 nt, Artificial Sequence
  EXO-Code
uagggaagagaaggacauaugau

SEQ ID NO: 2 - RNA, 23 nt, Artificial Sequence
  EXO-Code
uugacuaguacaugaccacuuga

SEQ ID NO: 3 - RNA, 25 nt, Artificial Sequence
  EXO-Code
acccugccgccuggacuccgccugu

SEQ ID NO: 4 - RNA, 20 nt, Artificial Sequence
  EXO-Code
gcagugaaagggacgguugc

SEQ ID NO: 5 - RNA, 20 nt, Artificial Sequence
  EXO-Code
uguguguccccacagcagug

SEQ ID NO: 6 - RNA, 20 nt, Artificial Sequence
  EXO-Code
cacucgcgagucgcggcagg

SEQ ID NO: 7 - RNA, 20 nt, Artificial Sequence
  EXO-Code
ugauguauugguaaguuucg

SEQ ID NO: 8 - RNA, 20 nt, Artificial Sequence
  EXO-Code
uuucguguuuagcguacaca

SEQ ID NO: 9 - RNA, 20 nt, Artificial Sequence
  EXO-Code
gguccaagccgagucgugcg

SEQ ID NO: 10 - RNA, 20 nt, Artificial Sequence
  EXO-Code
cugggaauuggcgugcguga

SEQ ID NO: 11 - RNA, 20 nt, Artificial Sequence
  EXO-Code
aaguuaauaucacacaucuc

SEQ ID NO: 12 - RNA, 20 nt, Artificial Sequence
  EXO-Code
gcuucaucaucucggggcac

SEQ ID NO: 13 - RNA, 20 nt, Artificial Sequence
  EXO-Code
uagcagagcacuuccucaca

SEQ ID NO: 14 - RNA, 20 nt, Artificial Sequence
  EXO-Code
gaggccugugcuaguaguga

SEQ ID NO: 15 - RNA, 20 nt, Artificial Sequence
  EXO-Code
ccggacaagugcgguuuguc

SEQ ID NO: 16 - RNA, 20 nt, Artificial Sequence
  EXO-Code
acaaucuccggcccacgaga

SEQ ID NO: 17 - RNA, 20 nt, Artificial Sequence
  EXO-Code
gcggguuauggaagccgguc

SEQ ID NO: 18 - RNA, 20 nt, Artificial Sequence
  EXO-Code
gggcaaugcugaagcgucgg

SEQ ID NO: 19 - RNA, 20 nt, Artificial Sequence
  EXO-Code
guuguacaugcacacaucuc

SEQ ID NO: 20 - RNA, 20 nt, Artificial Sequence
  EXO-Code
acaaucuccgacccacgaga

SEQ ID NO: 21 - RNA, 20 nt, Artificial Sequence
  EXO-Code
agugaguagcagaugcuaug

SEQ ID NO: 22 - RNA, 20 nt, Artificial Sequence
  EXO-Code
caucaaguuacgcccgcgca

SEQ ID NO: 23 - RNA, 20 nt, Artificial Sequence
  EXO-Code
gcgcggucucguuggcauag

SEQ ID NO: 24 - RNA, 20 nt, Artificial Sequence
  EXO-Code
guauugauuaaaagccacac

SEQ ID NO: 25 - RNA, 20 nt, Artificial Sequence
  EXO-Code
cuaggauggguacugccugc

SEQ ID NO: 26 - RNA, 20 nt, Artificial Sequence
  EXO-Code
gcgaauugacgugguuggcg

SEQ ID NO: 27 - RNA, 20 nt, Artificial Sequence
  EXO-Code
agcuuggcaagcugccgcug

SEQ ID NO: 28 - RNA, 20 nt, Artificial Sequence
  EXO-Code
gggaggaaaugugagcgugg

SEQ ID NO: 29 - RNA, 20 nt, Artificial Sequence
  EXO-Code
aaguauuucuuuccacacau

SEQ ID NO: 30 - RNA, 20 nt, Artificial Sequence
  EXO-Code
agcggacaaaggcagagggg

SEQ ID NO: 31 - RNA, 20 nt, Artificial Sequence
  EXO-Code
acguguggcugaggggcagc

SEQ ID NO: 32 - RNA, 20 nt, Artificial Sequence
  EXO-Code
gaugcacgcguguucggccg

SEQ ID NO: 33 - RNA, 20 nt, Artificial Sequence
  EXO-Code
ggguauggcggacuauggcc

SEQ ID NO: 34 - RNA, 20 nt, Artificial Sequence
  EXO-Code
gcguggacgagggguaugug

SEQ ID NO: 35 - RNA, 20 nt, Artificial Sequence
  EXO-Code
gcgguuggcacggggccugg

SEQ ID NO: 36 - RNA, 20 nt, Artificial Sequence
  EXO-Code
ggucgggucgguugcacggg

SEQ ID NO: 37 - RNA, 20 nt, Artificial Sequence
  EXO-Code
gcuaggugcugccacuguuc

SEQ ID NO: 38 - RNA, 20 nt, Artificial Sequence
  EXO-Code
gucggccagggacgcgugcg

SEQ ID NO: 39 - RNA, 20 nt, Artificial Sequence
  EXO-Code
uuucauguauugguguggca

SEQ ID NO: 40 - RNA, 20 nt, Artificial Sequence
  EXO-Code
ccgucguuagccaauugcuc

SEQ ID NO: 41 - RNA, 20 nt, Artificial Sequence
  EXO-Code
cgggagcuggcugggcagug

SEQ ID NO: 42 - RNA, 20 nt, Artificial Sequence
  EXO-Code
gacguugggggugaguacag

SEQ ID NO: 43 - RNA, 20 nt, Artificial Sequence
  EXO-Code
gugcugcacgcuggggagug

SEQ ID NO: 44 - RNA, 20 nt, Artificial Sequence
  EXO-Code
gcguggcuugccgugguuac

SEQ ID NO: 45 - RNA, 20 nt, Artificial Sequence
  EXO-Code
gugucgcaaggcccuugcag

SEQ ID NO: 46 - RNA, 20 nt, Artificial Sequence
  EXO-Code
cugguaaaauggcugccgga

SEQ ID NO: 47 - RNA, 20 nt, Artificial Sequence
  EXO-Code
gcaccauuguaaggcggacg

SEQ ID NO: 48 - RNA, 20 nt, Artificial Sequence
  EXO-Code
ggggcgagggaggucguuug

SEQ ID NO: 49 - RNA, 20 nt, Artificial Sequence
  EXO-Code
gagguuuagguuggcgggcg

SEQ ID NO: 50 - RNA, 20 nt, Artificial Sequence
  EXO-Code
agcaccagggguggcggagg

SEQ ID NO: 51 - RNA, 20 nt, Artificial Sequence
  EXO-Code
cggaguuaggaugcgccgcg

SEQ ID NO: 52 - RNA, 20 nt, Artificial Sequence
  EXO-Code
aggcaacggggcgggagggc

SEQ ID NO: 53 - RNA, 20 nt, Artificial Sequence
  EXO-Code
uggagaacuggugugcugga

SEQ ID NO: 54 - RNA, 20 nt, Artificial Sequence
  EXO-Code
aaggccggugcuaguaguga

SEQ ID NO: 55 - RNA, 20 nt, Artificial Sequence
  EXO-Code
acacaucccgagcccacgag

SEQ ID NO: 56 - RNA, 20 nt, Artificial Sequence
  EXO-Code
uuucguguuuagcguacaca

SEQ ID NO: 57 - RNA, 20 nt, Artificial Sequence
  EXO-Code
aguacaugaccacacacauc

SEQ ID NO: 58 - RNA, 20 nt, Artificial Sequence
  EXO-Code
gaggccugugcuaguaguga

SEQ ID NO: 59 - RNA, 20 nt, Artificial Sequence
  EXO-Code
cuaguacaugaccacacaca

SEQ ID NO: 60 - RNA, 20 nt, Artificial Sequence
  EXO-Code
ugauguauugguaaguuucg

SEQ ID NO: 61 - RNA, 20 nt, Artificial Sequence
  EXO-Code
uguguguccccacagcagug

SEQ ID NO: 62 - RNA, 20 nt, Artificial Sequence
  EXO-Code
gaggccggugcuaguaguga

SEQ ID NO: 63 - RNA, 20 nt, Artificial Sequence
  EXO-Code
acacacuccggcccacgaga

SEQ ID NO: 64 - RNA, 20 nt, Artificial Sequence
  EXO-Code
ucauugugcugaaacacauc

SEQ ID NO: 65 - RNA, 20 nt, Artificial Sequence
  EXO-Code
gggggauggauggaggggcg

SEQ ID NO: 66 - RNA, 20 nt, Artificial Sequence
  EXO-Code
acacauccccgagcccacga

SEQ ID NO: 67 - RNA, 20 nt, Artificial Sequence
  EXO-Code
aguacaugaccacuugaaca

SEQ ID NO: 68 - RNA, 20 nt, Artificial Sequence
  EXO-Code
ccguggcguguuggacacau

SEQ ID NO: 69 - RNA, 20 nt, Artificial Sequence
  EXO-Code
uagcagagcacuuccucaca

SEQ ID NO: 70 - RNA, 20 nt, Artificial Sequence
  EXO-Code
ccaccuuugccggaugcccg

SEQ ID NO: 71 - RNA, 20 nt, Artificial Sequence
  EXO-Code
ggggggcuggguguccgugg

SEQ ID NO: 72 - RNA, 20 nt, Artificial Sequence
  EXO-Code
ggagggaaggagggugccgg

SEQ ID NO: 73 - RNA, 20 nt, Artificial Sequence
  EXO-Code
ugauaguggagagcgcgcgg

SEQ ID NO: 74 - RNA, 20 nt, Artificial Sequence
  EXO-Code
gggcgaaauuggcauggccg

SEQ ID NO: 75 - RNA, 20 nt, Artificial Sequence
  EXO-Code
ggaggggggagggggcggug

SEQ ID NO: 76 - RNA, 20 nt, Artificial Sequence
  EXO-Code
agagggagggggugucgugg

SEQ ID NO: 77 - RNA, 20 nt, Artificial Sequence
  EXO-Code
ugauguauuugguuugcaag

SEQ ID NO: 78 - RNA, 20 nt, Artificial Sequence
  EXO-Code
gaaugcuuguucagacacau

SEQ ID NO: 79 - RNA, 20 nt, Artificial Sequence
  EXO-Code
cuaguacaugaccacuugaa

SEQ ID NO: 80 - RNA, 20 nt, Artificial Sequence
  EXO-Code
aaggccggcgcuaguaguga

SEQ ID NO: 81 - RNA, 20 nt, Artificial Sequence
  EXO-Code
ggugcggcuguaguuccggg

SEQ ID NO: 82 - RNA, 20 nt, Artificial Sequence
  EXO-Code
caugaccacuugaacacauc

SEQ ID NO: 83 - RNA, 20 nt, Artificial Sequence
  EXO-Code
ggagggaggaggggcgcggg

SEQ ID NO: 84 - RNA, 20 nt, Artificial Sequence
  EXO-Code
agggggaggaggcgggaugg

SEQ ID NO: 85 - RNA, 20 nt, Artificial Sequence
  EXO-Code
ggaggcggugaggguugugg

SEQ ID NO: 86 - RNA, 20 nt, Artificial Sequence
  EXO-Code
gcgcgauauggagggacugc

SEQ ID NO: 87 - RNA, 20 nt, Artificial Sequence
  EXO-Code
gggaggaggguggcgcgcgg

SEQ ID NO: 88 - RNA, 20 nt, Artificial Sequence
  EXO-Code
ccucggacaugucuuggugc

SEQ ID NO: 89 - RNA, 20 nt, Artificial Sequence
  EXO-Code
uaggcuuugaaacagauugc

SEQ ID NO: 90 - RNA, 20 nt, Artificial Sequence
  EXO-Code
uagcggggagggaggcgcgg

SEQ ID NO: 91 - RNA, 21 nt, Artificial Sequence
  EXO-Code
cgaggguguguccugugggcu

SEQ ID NO: 92 - RNA, 20 nt, Artificial Sequence
  EXO-Code
guguggggcgaggcgguggg

SEQ ID NO: 93 - RNA, 20 nt, Artificial Sequence
  EXO-Code
guauugauuaaaagccacac

SEQ ID NO: 94 - RNA, 20 nt, Artificial Sequence
  EXO-Code
gguuaauuuuaugugucaac

SEQ ID NO: 95 - RNA, 20 nt, Artificial Sequence
  EXO-Code
uggauggcagucgucacgug

SEQ ID NO: 96 - RNA, 20 nt, Artificial Sequence
  EXO-Code
guuggggaugucuguguggg

SEQ ID NO: 97 - RNA, 20 nt, Artificial Sequence
  EXO-Code
cgauucuggcgcgauccugg

SEQ ID NO: 98 - RNA, 20 nt, Artificial Sequence
  EXO-Code
ggggaggacggggggcgugg

SEQ ID NO: 99 - RNA, 20 nt, Artificial Sequence
  EXO-Code
gggaggggggugguccguug

SEQ ID NO: 100 - RNA, 20 nt, Artificial Sequence
  EXO-Code
ugcgauguugugaguggccc

SEQ ID NO: 101 - RNA, 20 nt, Artificial Sequence
  EXO-Code
gugcguauguuguguggggg

SEQ ID NO: 102 - RNA, 20 nt, Artificial Sequence
  EXO-Code
ccggcgggguuuggggccug

SEQ ID NO: 103 - RNA, 20 nt, Artificial Sequence
  EXO-Code
gaugucuuaucgucaugugu

SEQ ID NO: 104 - RNA, 20 nt, Artificial Sequence
  EXO-Code
ugauguauuugguuugcaag

SEQ ID NO: 105 - RNA, 20 nt, Artificial Sequence
  EXO-Code
ugauguauugguggguuucg

SEQ ID NO: 106 - RNA, 20 nt, Artificial Sequence
  EXO-Code
uuucguguauccuaguugcu

SEQ ID NO: 107 - RNA, 20 nt, Artificial Sequence
  EXO-Code
uuauugcaucuguaguaguu

SEQ ID NO: 108 - RNA, 20 nt, Artificial Sequence
  EXO-Code
ugaaaugagacugguuuugc

SEQ ID NO: 109 - RNA, 20 nt, Artificial Sequence
  EXO-Code
ugaauuguacagaagcuuga

SEQ ID NO: 110 - RNA, 20 nt, Artificial Sequence
  EXO-Code
uugaaaugugcuguugcaga

SEQ ID NO: 111 - RNA, 20 nt, Artificial Sequence
  EXO-Code
uugauguacguguaaguuua

SEQ ID NO: 112 - RNA, 20 nt, Artificial Sequence
  EXO-Code
ugcuuguacauuguuuacuu

SEQ ID NO: 113 - RNA, 20 nt, Artificial Sequence
  EXO-Code
guauuuguuuugauugcugc

SEQ ID NO: 114 - RNA, 20 nt, Artificial Sequence
  EXO-Code
uacacaugaacauagugaca

SEQ ID NO: 115 - RNA, 20 nt, Artificial Sequence
  EXO-Code
augcauuaguuacucgaaug

SEQ ID NO: 116 - RNA, 20 nt, Artificial Sequence
  EXO-Code
auuuucguguacgaaugguu

SEQ ID NO: 117 - RNA, 20 nt, Artificial Sequence
  EXO-Code
gaugugcgaguuuuauauug

SEQ ID NO: 118 - RNA, 20 nt, Artificial Sequence
  EXO-Code
ucaugguaacuaacuuguug

SEQ ID NO: 119 - RNA, 20 nt, Artificial Sequence
  EXO-Code
augguaguagcaauuguaaa

SEQ ID NO: 120 - RNA, 20 nt, Artificial Sequence
  EXO-Code
uaauguagcaaaguuuuuua

SEQ ID NO: 121 - RNA, 20 nt, Artificial Sequence
  EXO-Code
ggggaggagggagcgcgcgg

SEQ ID NO: 122 - RNA, 20 nt, Artificial Sequence
  EXO-Code
ccagggaggacgggucgugg

SEQ ID NO: 123 - RNA, 20 nt, Artificial Sequence
  EXO-Code
uugacguacgguuaucuaua

SEQ ID NO: 124 - RNA, 20 nt, Artificial Sequence
  EXO-Code
ggagggaggaggggcgcggg

SEQ ID NO: 125 - RNA, 20 nt, Artificial Sequence
  EXO-Code
acaugugauugguugcagug

SEQ ID NO: 126 - RNA, 20 nt, Artificial Sequence
  EXO-Code
ggagggagaggagggcgcgg

SEQ ID NO: 127 - RNA, 20 nt, Artificial Sequence
  EXO-Code
gugcuauggaauuauauuga

SEQ ID NO: 128 - RNA, 20 nt, Artificial Sequence
  EXO-Code
ggggaggacggggggcgugg

SEQ ID NO: 129 - RNA, 20 nt, Artificial Sequence
  EXO-Code
aauuacacugugcuaggaug

SEQ ID NO: 130 - RNA, 20 nt, Artificial Sequence
  EXO-Code
gcauuuaugacuaagucuug

SEQ ID NO: 131 - RNA, 20 nt, Artificial Sequence
  EXO-code
gcugggggaggggcguuggg

SEQ ID NO: 132 - RNA, 20 nt, Artificial Sequence
  EXO-Code
gcgggaaggggggccugugg

SEQ ID NO: 133 - RNA, 20 nt, Artificial Sequence
  EXO-Code
gagguuggggagggccgugg

SEQ ID NO: 134 - RNA, 20 nt, Artificial Sequence
  EXO-Code
gggggauggauggaggggcg

SEQ ID NO: 135 - RNA, 20 nt, Artificial Sequence
  EXO-Code
ggaggggggagggggcgcgg

SEQ ID NO: 136 - RNA, 20 nt, Artificial Sequence
  EXO-Code
ggggcuggggcguggugugg

SEQ ID NO: 137 - RNA, 20 nt, Artificial Sequence
  EXO-Code
ggguagguggagggcguugg

SEQ ID NO: 138 - RNA, 20 nt, Artificial Sequence
  EXO-Code
acagacgguugcuugcgggg

SEQ ID NO: 139 - RNA, 20 nt, Artificial Sequence
  EXO-Code
gggggaagcguguucggugg

SEQ ID NO: 140 - RNA, 20 nt, Artificial Sequence
  EXO-Code
ggggggaggagggggcugcg

SEQ ID NO: 141 - RNA, 20 nt, Artificial Sequence
  EXO-Code
gcauguauugguuuuugguu

SEQ ID NO: 142 - RNA, 20 nt, Artificial Sequence
  EXO-Code
ugaagcuguacaaaguuugc

SEQ ID NO: 143 - RNA, 20 nt, Artificial Sequence
  EXO-Code
uagcagaacggcgcgugugg

SEQ ID NO: 144 - RNA, 20 nt, Artificial Sequence
  EXO-Code
gggcgcacauauguuggugg

SEQ ID NO: 145 - RNA, 20 nt, Artificial Sequence
  EXO-Code
gggggacgggcggggguugg

SEQ ID NO: 146 - RNA, 20 nt, Artificial Sequence
  EXO-Code
ucauugugcugaaacaaucu

SEQ ID NO: 147 - RNA, 20 nt, Artificial Sequence
  EXO-Code
ggggaaggaggcgggcgugg

SEQ ID NO: 148 - RNA, 20 nt, Artificial Sequence
  EXO-Code
ggggagggaaggcgggcgga

SEQ ID NO: 149 - RNA, 20 nt, Artificial Sequence
  EXO-Code
uuuguguacaaagcagauuc

SEQ ID NO: 150 - RNA, 20 nt, Artificial Sequence
  EXO-Code
aguguauugcgaucaguuga

SEQ ID NO: 151 - RNA, 20 nt, Artificial Sequence
  EXO-Code
ggggggugaggggggaccgg

SEQ ID NO: 152 - RNA, 20 nt, Artificial Sequence
  EXO-Code
cgcuggucugccugugugcg

SEQ ID NO: 153 - RNA, 20 nt, Artificial Sequence
  EXO-Code
gcacaaucuccgagcccacg

SEQ ID NO: 154 - RNA, 20 nt, Artificial Sequence
  EXO-Code
uuucguguuuagcguacaca

SEQ ID NO: 155 - RNA, 20 nt, Artificial Sequence
  EXO-Code
ugauguauugguaaguuucg

SEQ ID NO: 156 - RNA, 20 nt, Artificial Sequence
  EXO-Code
aacuguauugguuauacaca

SEQ ID NO: 157 - RNA, 20 nt, Artificial Sequence
  EXO-Code
uauagaugugcuaguuugca

SEQ ID NO: 158 - RNA, 20 nt, Artificial Sequence
  EXO-Code
aacaaucuccgagcccacga

SEQ ID NO: 159 - RNA, 20 nt, Artificial Sequence
  EXO-Code
uuucguguuuggcguacaca

SEQ ID NO: 160 - RNA, 20 nt, Artificial Sequence
  EXO-Code
ucauugugcugaaacacauc

SEQ ID NO: 161 - RNA, 20 nt, Artificial Sequence
  EXO-Code
ugauguauugguaaguuuug

SEQ ID NO: 162 - RNA, 20 nt, Artificial Sequence
  EXO-Code
aacuguauugguuguacaca

SEQ ID NO: 163 - RNA, 20 nt, Artificial Sequence
  EXO-Code
aaggccggugcuaguaguga

SEQ ID NO: 164 - RNA, 20 nt, Artificial Sequence
  EXO-Code
uuucguguuuagcgacacau

SEQ ID NO: 165 - RNA, 20 nt, Artificial Sequence
  EXO-Code
uuucguguuuagcguacaau

SEQ ID NO: 166 - RNA, 20 nt, Artificial Sequence
  EXO-Code
uuucguguuuagcuuacaca

SEQ ID NO: 167 - RNA, 20 nt, Artificial Sequence
  EXO-Code
acguguauugcuaacacauc

SEQ ID NO: 168 - RNA, 20 nt, Artificial Sequence
  EXO-Code
acauguauugguuuuugguu

SEQ ID NO: 169 - RNA, 20 nt, Artificial Sequence
  EXO-Code
ugauguauuggugaguuucg

SEQ ID NO: 170 - RNA, 20 nt, Artificial Sequence
  EXO-Code
uuuuguguacuugcauuuca

SEQ ID NO: 171 - RNA, 20 nt, Artificial Sequence
  EXO-Code
gcguguauuacuaacacauc

SEQ ID NO: 172 - RNA, 20 nt, Artificial Sequence
  EXO-Code
ugauguauuugguuugcaag

SEQ ID NO: 173 - RNA, 20 nt, Artificial Sequence
  EXO-Code
ugauguauugguagguuucg

SEQ ID NO: 174 - RNA, 20 nt, Artificial Sequence
  EXO-Code
cacacaaucuccgagcccac

SEQ ID NO: 175 - RNA, 20 nt, Artificial Sequence
  EXO-Code
agcuguauugguuauacaca

SEQ ID NO: 176 - RNA, 20 nt, Artificial Sequence
  EXO-Code
acaugugauuaguugcaaug

SEQ ID NO: 177 - RNA, 20 nt, Artificial Sequence
  EXO-Code
uauagaugugcugguuugca

SEQ ID NO: 178 - RNA, 20 nt, Artificial Sequence
  EXO-Code
aacacauuccgagcccacga

SEQ ID NO: 179 - RNA, 20 nt, Artificial Sequence
  EXO-Code
ugaagcuguacaaaguuugu

SEQ ID NO: 180 - RNA, 20 nt, Artificial Sequence
  EXO-Code
acaugugauugguugcaaug

SEQ ID NO: 181 - RNA, 20 nt, Artificial Sequence
  EXO-Code
agcuguauugguuguacaca

SEQ ID NO: 182 - RNA, 20 nt, Artificial Sequence
  EXO-Code
gcguguauugcuaacacauc

SEQ ID NO: 183 - RNA, 20 nt, Artificial Sequence
  EXO-Code
ugauguauugguggguuucg

SEQ ID NO: 184 - RNA, 20 nt, Artificial Sequence
  EXO-Code
cugaugucucuuauacagac

SEQ ID NO: 185 - RNA, 20 nt, Artificial Sequence
  EXO-Code
aacacucuccgagcccacga

SEQ ID NO: 186 - RNA, 20 nt, Artificial Sequence
  EXO-Code
uuugcaaguguacaguuguu

SEQ ID NO: 187 - RNA, 20 nt, Artificial Sequence
  EXO-Code
ugauguguuaguuugaaugu

SEQ ID NO: 188 - RNA, 20 nt, Artificial Sequence
  EXO-Code
ggggggcuggguguccgugg

SEQ ID NO: 189 - RNA, 20 nt, Artificial Sequence
  EXO-Code
uuucguguuuagcguacauc

SEQ ID NO: 190 - RNA, 20 nt, Artificial Sequence
  EXO-Code
uuucguguuuagcguacacu

SEQ ID NO: 191 - RNA, 20 nt, Artificial Sequence
  EXO-Code
aacacaucccgagcccacga

SEQ ID NO: 192 - RNA, 20 nt, Artificial Sequence
  EXO-Code
acguguauuacuaacaaucu

SEQ ID NO: 193 - RNA, 20 nt, Artificial Sequence
  EXO-Code
ugaauguagcuuaguacaaa

SEQ ID NO: 194 - RNA, 20 nt, Artificial Sequence
  EXO-Code
gaaauguacaaugaucacac

SEQ ID NO: 195 - RNA, 20 nt, Artificial Sequence
  EXO-Code
ggggaggacggggggcgugg

SEQ ID NO: 196 - RNA, 20 nt, Artificial Sequence
  EXO-Code
aacacauccgagcccacgag

SEQ ID NO: 197 - RNA, 20 nt, Artificial Sequence
  EXO-Code
aacaacuccgagcccacgag

SEQ ID NO: 198 - RNA, 20 nt, Artificial Sequence
  EXO-Code
aacacacuccggcccacgag

SEQ ID NO: 199 - RNA, 20 nt, Artificial Sequence
  EXO-Code
ugacugucucuuauacagac

SEQ ID NO: 200 - RNA, 20 nt, Artificial Sequence
  EXO-Code
uuacaaugcgcuaguuuuug

SEQ ID NO: 201 - RNA, 20 nt, Artificial Sequence
  EXO-Code
aacacauccccgagcccacg

SEQ ID NO: 202 - RNA, 20 nt, Artificial Sequence
  EXO-Code
cugcugucucuuauacagac

SEQ ID NO: 203 - RNA, 20 nt, Artificial Sequence
  EXO-Code
acguguauuacuaacacacu

SEQ ID NO: 204 - RNA, 20 nt, Artificial Sequence
  EXO-Code
uugaaguguacauugucgua

SEQ ID NO: 205 - RNA, 20 nt, Artificial Sequence
  EXO-Code
uuauugcaucuguaguaguu

SEQ ID NO: 206 - RNA, 20 nt, Artificial Sequence
  EXO-Code
uaauguauuggcauaacuac

SEQ ID NO: 207 - RNA, 20 nt, Artificial Sequence
  EXO-Code
gauguauaguuuugaugcac

SEQ ID NO: 208 - RNA, 20 nt, Artificial Sequence
  EXO-Code
gagggaggaggagggcggcg

SEQ ID NO: 209 - RNA, 20 nt, Artificial Sequence
  EXO-Code
aacaccucuccgagcccacg

SEQ ID NO: 210 - RNA, 20 nt, Artificial Sequence
  EXO-Code
gacaaucuccgagcccacga

SEQ ID NO: 211 - RNA, 20 nt, Artificial Sequence
  EXO-Code
ugauguauuggugaguuuug

SEQ ID NO: 212 - RNA, 20 nt, Artificial Sequence
  EXO-Code
aacuguauugguuauacaau

SEQ ID NO: 213 - RNA, 20 nt, Artificial Sequence
  EXO-Code
ggggaaggaggcgggcgugg

SEQ ID NO: 214 - RNA, 20 nt, Artificial Sequence
  EXO-Code
guauuguaguaauuacugua

SEQ ID NO: 215 - RNA, 20 nt, Artificial Sequence
  EXO-Code
uuuuguguuuagcguacaca

SEQ ID NO: 216 - RNA, 20 nt, Artificial Sequence
  EXO-Code
ugauugauacuguguaauua

SEQ ID NO: 217 - RNA, 20 nt, Artificial Sequence
  EXO-Code
ugauaguauuggucuauuca

SEQ ID NO: 218 - RNA, 20 nt, Artificial Sequence
  EXO-Code
uugauguacguguaaguuua

SEQ ID NO: 219 - RNA, 20 nt, Artificial Sequence
  EXO-Code
uuucgcguuuagcguacaca

SEQ ID NO: 220 - RNA, 20 nt, Artificial Sequence
  EXO-Code
aacauaucuccgagcccacg

SEQ ID NO: 221 - RNA, 20 nt, Artificial Sequence
  EXO-Code
auaacaaacugugcuagaca

SEQ ID NO: 222 - RNA, 20 nt, Artificial Sequence
  EXO-Code
aacaaucuccgacccacgag

SEQ ID NO: 223 - RNA, 20 nt, Artificial Sequence
  EXO-Code
ugauguauugguagguuuug

SEQ ID NO: 224 - RNA, 20 nt, Artificial Sequence
  EXO-Code
ggggggaggagggggcugcg

SEQ ID NO: 225 - RNA, 20 nt, Artificial Sequence
  EXO-Code
cgcacaaucuccgagcccac

SEQ ID NO: 226 - RNA, 20 nt, Artificial Sequence
  EXO-Code
gggcugggggggggccgugg

SEQ ID NO: 227 - RNA, 20 nt, Artificial Sequence
  EXO-Code
uuucguguuuagcauacaca

SEQ ID NO: 228 - RNA, 20 nt, Artificial Sequence
  EXO-Code
uuucguguuuaucguacaca

SEQ ID NO: 229 - RNA, 20 nt, Artificial Sequence
  EXO-Code
uuucgugcuuagcguacaca

SEQ ID NO: 230 - RNA, 20 nt, Artificial Sequence
  EXO-Code
ccagggaggacgggucgugg

SEQ ID NO: 231 - RNA, 20 nt, Artificial Sequence
  EXO-Code
cuagcgacggugcgggggug

SEQ ID NO: 232 - RNA, 20 nt, Artificial Sequence
  EXO-Code
cuucguguuuagcguacaca

SEQ ID NO: 233 - RNA, 20 nt, Artificial Sequence
  EXO-Code
ucauggauacuaugcauuga

SEQ ID NO: 234 - RNA, 20 nt, Artificial Sequence
  EXO-Code
aacaaucuccggcccacgag

SEQ ID NO: 235 - RNA, 20 nt, Artificial Sequence
  EXO-Code
acguguauuacugacacauc

SEQ ID NO: 236 - RNA, 20 nt, Artificial Sequence
  EXO-Code
ugauguuacuguuguuucga

SEQ ID NO: 237 - RNA, 20 nt, Artificial Sequence
  EXO-Code
uuucguguuuagcgcacaca

SEQ ID NO: 238 - RNA, 20 nt, Artificial Sequence
  EXO-Code
ugauguauuugguuugcagg

SEQ ID NO: 239 - RNA, 20 nt, Artificial Sequence
  EXO-Code
uuucguguucagcguacaca

SEQ ID NO: 240 - RNA, 20 nt, Artificial Sequence
  EXO-Code
ggggaggauauggccugugg

SEQ ID NO: 241 - RNA, 20 nt, Artificial Sequence
  EXO-Code
ccguggggaggggagcucgg

SEQ ID NO: 242 - RNA, 20 nt, Artificial Sequence
  EXO-Code
aguguauugugucauacuga

SEQ ID NO: 243 - RNA, 20 nt, Artificial Sequence
  EXO-Code
gggcgggaaucguggugcgg

SEQ ID NO: 244 - RNA, 20 nt, Artificial Sequence
  EXO-Code
ggagggaggaggggcgcggg

SEQ ID NO: 245 - RNA, 20 nt, Artificial Sequence
  EXO-Code
uuauguaauggcgauuuaca

SEQ ID NO: 246 - RNA, 20 nt, Artificial Sequence
  EXO-Code
gagguuggggagggccgugg

SEQ ID NO: 247 - RNA, 20 nt, Artificial Sequence
  EXO-Code
uaucaugguacaguuuuggc

SEQ ID NO: 248 - RNA, 20 nt, Artificial Sequence
  EXO-Code
gacuguauugguuauacaca

SEQ ID NO: 249 - RNA, 20 nt, Artificial Sequence
  EXO-Code
gcacugguuuguaacacauc

SEQ ID NO: 250 - RNA, 20 nt, Artificial Sequence
  EXO-Code
ggaggggggagggggcgcgg

SEQ ID NO: 251 - RNA, 20 nt, Artificial Sequence
  EXO-Code
uuucguguuuagcguacaac

SEQ ID NO: 252 - RNA, 20 nt, Artificial Sequence
  EXO-Code
ugcuuguacauuguuuacuu

SEQ ID NO: 253 - RNA, 20 nt, Artificial Sequence
  EXO-Code
ccguggguggcugggugugg

SEQ ID NO: 254 - RNA, 20 nt, Artificial Sequence
  Random control EXO-Code
acacaucuccgagcccacga

SEQ ID NO: 255 - RNA, 12 nt, Artificial Sequence
  Discovered EXO-Code motif #1 IUPAC nucleotide code
guacmygacsac

SEQ ID NO: 256 - RNA, 12 nt, Artificial Sequence
  Disocvered EXO-Code motif #1 sequence example
guacaugaccac

SEQ ID NO: 257 - RNA, 12 nt, Artificial Sequence
  Discovered EXO-Code motif #1 sequence example
guacccgacgac

SEQ ID NO: 258 - RNA, 12 nt, Artificial Sequence
  Discovered EXO-Code motif #2 IUPAC nucleotide code
wsvuguryursu

SEQ ID NO: 259 - RNA, 12 nt, Artificial Sequence
  Discovered EXO-Code motif #2 sequence example
ugauguauuggu

SEQ ID NO: 260 - RNA, 12 nt, Artificial Sequence
  Discovered EXO-Code motif #2 sequence example
acgugugcuacu

SEQ ID NO: 261 - RNA, 12 nt, Artificial Sequence
  Discovered EXCO-Code motif #3 IUPAC nucleotide code
grgaaggacrum

SEQ ID NO: 262 - RNA, 12 nt, Artificial Sequence
  Discovered EXO-Code motif #3 sequence example
gggaaggacguc

SEQ ID NO: 263 - RNA, 12 nt, Artificial Sequence
  Discovered EXO-Code motif #3 sequence example
gagaaggacaua

SEQ ID NO: 264 - RNA, 12 nt, Artificial Sequence
  Discovered EXO-Code motif #4 IUPAC sequence code
gucacacagucc

(19) United States
(12) Patent Application Publication
(10) Pub. No.: US 2022/0372480 A1
(43) Pub. Date: Nov. 24, 2022

(54) COMPOSITIONS AND METHODS FOR LOADING EXTRACELLULAR VESICLES WITH CHEMICAL AND BIOLOGICAL AGENTS/MOLECULES

(71) Applicants: The Research Foundation for The State University of New York, Amherst, NY (US); The University of North Carolina at Chapel Hill, Chapel Hill, NC (US)

(72) Inventors: Juliane NGUYEN, Buffalo, NY (US); Scott FERGUSON, North Tonawanda, NY (US); Emily BONACQUISTI, Chapel Hill, NC (US)

(21) Appl. No.: 17/772,505

(22) PCT Filed: Oct. 30, 2020
(86) PCT No.: PCT/US2020/058210

§ 371 Date: Apr. 27, 2022

Related U.S. Application Data
(60) Provisional application No. 62/928,637, filed on Oct. 31, 2019.

(51) Int. Cl. C12N 15/113; C12N 15/11

(52) U.S. Cl. CPC C12N 15/113; C12N 15/111; C12N 2310/11; C12N 2310/14; C12N 2310/10

(57) ABSTRACT

Provided are RNA polynucleotide sequences referred to as “EXO-Codes.” The RNA polynucleotides comprise one or more nucleotide sequences that facilitate preferential enrichment of secreted membranous vesicles that contain the EXO-Codes. Nucleotide motifs that contribute to these properties of the EXO-Codes are described. Modified eukaryotic cells comprising EXO-Codes are provided, and include lymphocytes such as T cells. EXO-Codes may comprise a cargo that provides a prophylactic or therapeutic effect. Exosome preparations comprising the EXO-codes are provided. Pharmaceutical compositions comprising EXO-Codes, expression vectors encoding them, and methods of using the pharmaceutical formulations are provided. The pharmaceutical compositions may comprise the EXO-Codes within membranous structures, such as exosomes. Cells that express or otherwise include the EXO-Codes are included, as are method for separating membranous structures that contain the EXO-codes from the cells.

### CROSS-REFERENCE TO RELATED APPLICATIONS

This application claims priority to U.S. provisional application No. 62/928,637, filed Oct. 31, 2019, the disclosure of which is incorporated herein by reference.

### STATEMENT REGARDING FEDERALLY SPONSORED RESEARCH OR DEVELOPMENT

This invention was made with government support under grant number R01EB023262 awarded by National Institutes of Health. The government has certain rights in the invention.

### FIELD

The present disclosure relates generally to compositions and methods for loading exosomes/extracellular vesicles with chemical and biological agents and/or for modulating cargo sorting to exosomes/extracellular vesicles, wherein the chemical or biological agents are preferentially enriched in the exosomes/extracellular vesicles in lymphocytes, such as T cells.

### BACKGROUND

In a milliliter of human blood, there are roughly one billion exosomes: lipid vesicles that contain proteins, miRNA, and mRNA. Cells release exosomes into the extracellular environment where they participate in a number of physiological and pathophysiological processes. Exosomes originate from multivesicular bodies (MVBs), which fuse with the plasma cell membrane before release into the extracellular environment. Upon release, exosomes can fuse with neighboring cells to transfer their cargo. It is now known that exosomes are not merely vehicles for unwanted cellular proteins and ‘junk RNA’ but that cells actively secrete exosomes to modulate their microenvironment.

Exosomes are involved in the pathogenesis of several diseases including cancer, neurodegenerative, autoimmune, and liver diseases. Several studies have examined the role of exosomes in cancer growth and metastasis [Costa-Silva B, et al. Nat Cell Biol. 2015; 17(6):816-826; Grange C, et al. Cancer research. 2011; 71(15):5346-5356; Hood J L, et al. Cancer research. 2011; 71(11):3792-3801; Kucharzewska P, et al. Proceedings of the National Academy of Sciences of the United States of America. 2013; 110(18):7312-7317; Peinado H, et al. Nat Med. 2012; 18(6):883-891]. Since extracellular vesicles such as exosomes derived from tumor cells have the potential to convert adipose-derived mesenchymal stem cells into tumor-associated myofibroblasts, it has been proposed that these exosomes can contribute to tumor progression and the malignant phenotype by generating tumor stroma [Cho J A, et al. Int J Oncol. 2012; 40(1):130-138]. Exosomes also play a role in inducing metastases, which are ultimately responsible for over 90% of cancer-related deaths: the treatment of metastatic disease remains a clinical challenge. Cancer cell-derived exosomes have the potential to convert healthy cells into tumor-forming cells in their immediate microenvironment: exosomes released by cancer cells can transfer onco-genes (mainly via oncogenic small RNAs) to recipient cells, induce migration of cancer cells, and promote angiogenesis, which are critical cancer “hallmarks” [Meehan K, et al. Crit Rev Clin Lab Sci. 2015:1-11]. Due to their inherent ability for systemic spread, they can also initiate new tumor growth at distant sites by preparing a pre-metastatic niche. In particular, it has been shown how exosomes from lung-tropic 4175-LuT cancer cells (a MDA-MB-231 breast cancer sub-line) specifically located to the lung and were taken up by lung-resident fibroblasts after systemic administration. These exosomes were able to not only redirect the migration of bone-tropic tumor cells from bone sites to the lung but also increased the metastatic capacity of those cells in the lung by 10,000 fold [Hoshino A, et al. Nature. 2015; 527(7578):329-335]]. These data indicate that circulating exosomes prepare discrete sites for future metastatic tumors consistent with the seed-soil hypothesis. Exosomes and/or extracellular vesicles also play a role in physiological processes and can have regenerative effects. For example, exosomes/extracellular vesicles derived from mesenchymal stem cells (MSCs) have been shown to mediate cardiac tissue repair after myocardial infarction by modulating the injured tissue environment, inducing angiogenesis, and inducing cellular proliferation and differentiation [Barile L et al. Cardiovasc Res. 2014; 103(4):530-541; Lai R C. et al. Stem cell research. 2010; 4(3):214-222]. However, there is an ongoing and unmet need for new and improved compositions and methods that are useful for reprogramming exosomes, such as those that can be secreted by T cells, to halt and/or reverse disease progression, and for reprogramming exosomes so that they can deliver other desirable cargo to target cells. The present disclosure is pertinent to these needs.

### BRIEF SUMMARY

The present disclosure provides RNA polynucleotide sequences referred to herein as “EXO-Codes.” The RNA polynucleotide comprise one or more nucleotide sequences that facilitate preferential enrichment of secreted membranous vesicles, such as exosomes, that contain the EXO-Codes. Nucleotide motifs that contribute to these properties of the EXO-Codes are described. In embodiments, the motifs include at least one of: GUACMYGACSAC (SEQ ID NO: 255), WSVUGURYURSU (SEQ ID NO: 258), GRGAAGGACRUM (SEQ ID NO: 261), or GUCACACAGUCC (SEQ ID NO: 264). These representative sequences are provided using IUPAC nucleotide nomenclature. Thus, in the described sequences, M is A or C; Y is C or U; S is C or G; W is A or U; V is A or C or G; and R is A or G. The disclosure includes every nucleotide sequence that meets this definition in the context of the described sequences. Additional EXO-Code sequences are provided the Tables, non-limiting examples of which comprise GGAGGGAGGAGGGGCGCGGG (SEQ ID NO:91) (“T2:); ACAUGUAUUGGUUUUUGGUU (SEQ ID NO:168) (“T3”); and UUUCGUGUUUAGCGUACACA (SEQ ID NO:154 (“T4”).

The disclosure includes modified eukaryotic cells comprising the described EXO-Codes. The eukaryotic cells include but are not necessarily limited to lymphocytes, such as T cells.

The disclosure includes modifying eukaryotic cells such that the comprise EXO-Codes. The cells may comprise the EXO-Codes by direct introduction of RNA into the cells, or by introduction of an expression vector that encodes an EXO-Code.

The disclosure includes providing RNA polynucleotides that include an EXO-Code and may also comprise a cargo. The cargo is not particularly limited, and may be a cargo that provides a prophylactic or therapeutic effect. The cargo may be part of the RNA polynucleotide that comprises the EXO-Code, or it may be provided in combination with the RNA polynucleotide. In embodiments, the RNA polynucleotide that comprises the EXO-Code may function as an aptamer.

Pharmaceutical compositions comprising RNA polynucleotides, and expression vectors encoding them, are included for use in prophylaxis or therapy of a variety of diseases, non-limiting examples of which include cancers and autoimmune disorders. The pharmaceutical compositions may comprise the EXO-Codes within membranous structures, such as exosomes. Cells that express or otherwise include the EXO-Codes may also be used for therapeutic and prophylactic purposes. Such cells themselves are encompassed by the disclosure.

The disclosure also includes isolated EXO-Code containing RNA polynucleotides, cDNAs of the RNA polynucleotides, and expression vectors that encode the RNA polynucleotides.

The disclosure also includes methods of making membranous structures such as exosomes that comprise EXO-Codes by programming cells to produce and secrete the described structures, and separating the described structures from the cells.

### BRIEF DESCRIPTION OF THE FIGURES

FIG. 1. Schematic of an RNA library screening approach used to identify EXO-Codes of this disclosure. Cells are electroporated with a large pool of RNA sequences (1012 diversity). Exosomes are collected and the RNA sequences extracted. After conversion into cDNA, PCR amplified DNA is transcribed into RNA to create the RNA pool used for the next rounds of selection. Selection rounds are repeated 6 times with increased selection pressure (by reducing the amount of input RNA). Illumina next-generation sequencing was performed for selection rounds 3 to 7 in human T cells. T lymphocytes (T cells) were isolated from human donor blood using Corning Lymphocyte Separation Medium (Cat #25-072-CV) according the manufacturer's protocol. The T cells were activated with anti-CD3 antibody (Anti-human CD3 functional grade, Cat #16-0037-85, eBioscience) for 48 h. T cells were then cultured in RPMI containing 10% of heat-inactivated fetal bovine serum, Pen Strep and IL-2 (final conc. 300 IU/mL).

FIG. 2. Enriched EXO-Codes in human T cells (donor 1). Identified EXO-Codes are significantly higher enriched in exosomes than a random control. EXO-Code T2 is ˜27,100-fold more enriched in exosomes than the random control sequence. EXO-Code T4 is ˜285-fold more enriched in exosomes than random control sequence. Statistical analysis was performed with one-way ANOVA followed by Dunnett's post-hoc test (****p<0.0001).

FIG. 3. Enriched EXO-Codes in human T cells (donor 2). Identified EXO-Codes are significantly higher enriched in exosomes than a random control. EXO-Code T2 is ˜2,413-fold more enriched in exosomes than the random control sequence. EXO-Code T4 is ˜40-fold more enriched in exosomes than random control sequence. Statistical analysis was performed with one-way ANOVA followed by Dunnett's post-hoc test (****p<0.0001).

FIG. 4. EXO-Code motifs and corresponding IUPAC nucleotide codes. EXO-Code motif #1 has an IUPAC nucleotide code of GUACMYGACSAC (SEQ ID NO: 255) and includes, but is not limited to, the sequences GUACAUGACCAC (SEQ ID NO: 256) and GUACCCGACGAC (SEQ ID NO: 257). EXO-Code motif #2 has an IUPAC nucleotide code of WSVUGURYURSU (SEQ ID NO: 258) and includes, but is not limited to, the sequences UGAUGUAUUGGU (SEQ ID NO: 259) and ACGUGUGCUACU (SEQ ID NO: 260). EXO-Code motif #3 has an IUPAC nucleotide code of GRGAAGGACRUM (SEQ ID NO: 261) and includes, but is not limited to, the sequences GGGAAGGACGUC (SEQ ID NO: 262) and GAGAAGGACAUA (SEQ ID NO: 263). EXO-Code motif #4 has an IUPAC nucleotide code of GUCACACAGUCC (SEQ ID NO: 264) and includes the sequence GUCACACAGUCC (SEQ ID NO: 264). IUPAC nucleotide symbol meanings for described EXO-Code sequences include M=A or C; Y=C or U; S=C or G; W=A or U; V=A or C or G; and R=A or G.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. The disclosure includes polynucleotide consensus sequences and motifs. Each nucleotide sequence of this disclosure may comprise or consist of the described sequence.

The present disclosure relates to RNA polynucleotide sequences (referred to herein from time to time as “EXO-Codes”) that are capable of a) selectively sorting to extracellular vesicles such as exosomes, and b) delivering a variety of cargo types to program or reprogram the extracellular vesicles. While non-limiting embodiments of the disclosure are illustrated using exosomes, the disclosure includes using EXO-Codes to sort polynucleotides containing the EXO-Codes to any secreted membranous structures, including but not necessarily limited to exosomes, vesicles, microvesicles, micro-particles, endosomal derived vesicles, multivesicular bodies, apoptotic bodies, and combinations thereof.

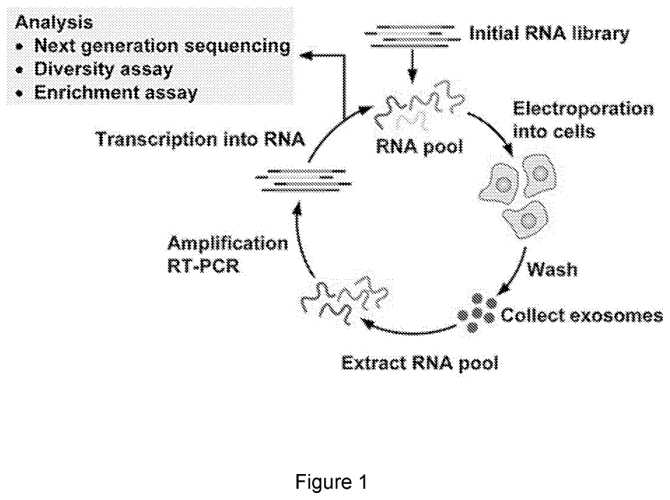

FIG. 1 provides an overview of a process by which EXO-Codes of this disclosure were identified. The tables of this disclosure provide representative and non-limiting EXO-Code sequences. In part from the identified EXO-Code sequences, the disclosure provides EXO-Code motifs. Thus, in embodiments, an EXO-Code of this disclosure comprises a motif sequence. The motif sequence facilitates preferential enrichment of membranous vesicles with the EXO-Codes within T cells, relative to enrichment of membranous vesicles by the T cells with a control RNA polynucleotide.

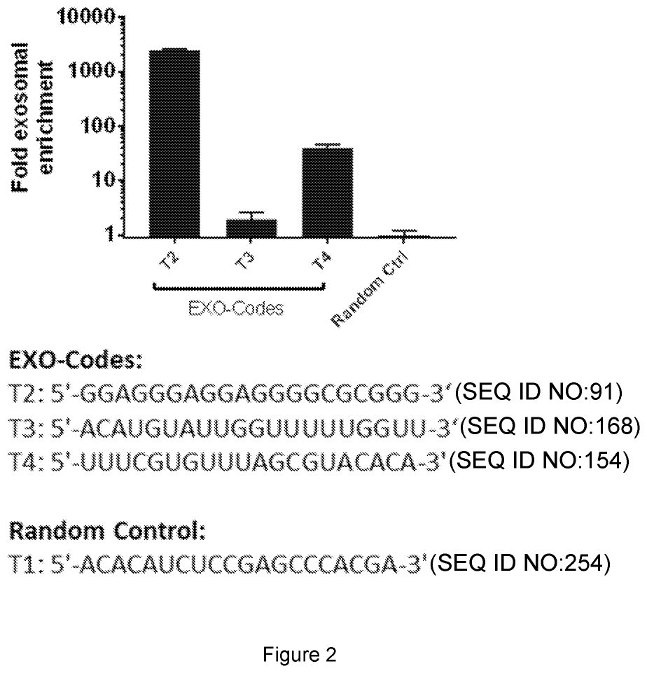

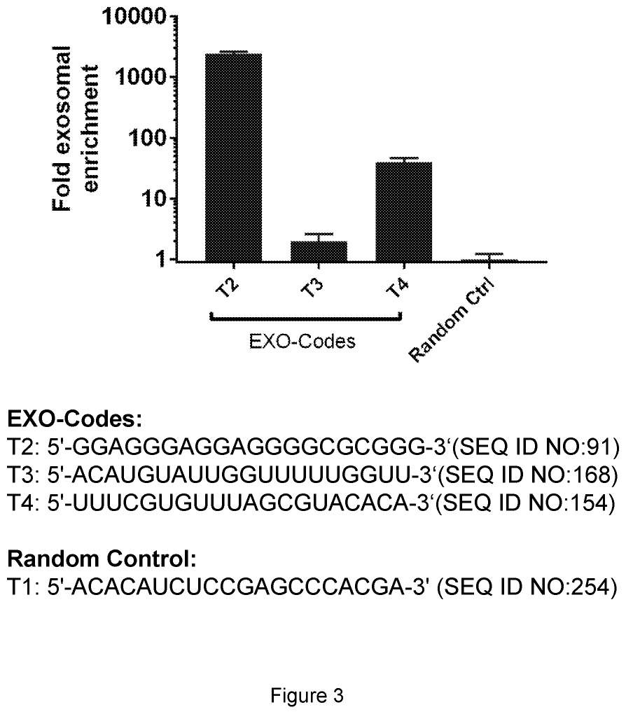

The disclosure includes all motif sequences and all sequences having one or more of the alternative nucleotides in the particular motif sequences, and all combinations of such alternative nucleotides. Non-limiting embodiments of specific EXO-Codes sequences are depicted in FIGS. 1-3. These representative sequences comprise GGAGGGAGGAGGGGCGCGGG (SEQ ID NO:91) (“T2:); ACAUGUAUUGGUUUUUGGUU (SEQ ID NO:168) (“T3”); and UUUCGUGUUUAGCGUACACA (SEQ ID NO:154 (“T4”). FIGS. 1, 2 and 3 describe enrichment of the described sequences in exosomes, relative to enrichment of a randomized control RNA polynucleotide.

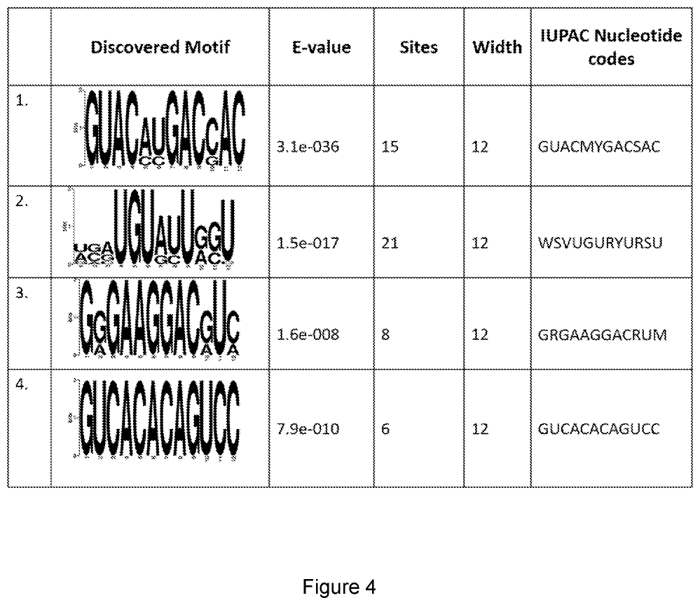

In embodiments, a motif sequence of this disclosure is represented using International Union of Pure and Applied Chemistry (IUPAC) nucleotide symbols, which are known in the art to be as follows: M=A or C; Y=C or U; S=C or G; W=A or U; V=A or C or G; and R=A or G. In embodiments, a motif of the disclosure comprises a sequence referred to herein as EXO-Code motif #1, which comprises the sequence GUACMYGACSAC (SEQ ID NO: 255), non-limiting examples of which include the sequences GUACAUGACCAC (SEQ ID NO: 256) and GUACCCGACGAC (SEQ ID NO: 257), as shown, for example, in FIG. 4. EXO-Code motif #2 comprises the sequence WSVUGURYURSU (SEQ ID NO: 258), non-limiting examples of which include the sequences UGAUGUAUUGGU (SEQ ID NO: 259) and ACGUGUGCUACU (SEQ ID NO: 260), as shown in FIG. 4. EXO-Code motif #3 comprises the sequence GRGAAGGACRUM (SEQ ID NO: 261), non-limiting examples of which include the sequences GGGAAGGACGUC (SEQ ID NO: 262) and GAGAAGGACAUA (SEQ ID NO: 263), as shown in FIG. 4. EXO-Code motif #4 comprises the sequence GUCACACAGUCC (SEQ ID NO: 264), a non-limiting example of which includes the sequence GUCACACAGUCC (SEQ ID NO: 264). To obtain the candidate EXO-Codes and motif sequences, human T-cells were electroporated with a large pool of RNA sequences. Exosomes were collected and the RNA sequences extracted. After conversion into cDNA, PCR amplified DNA was transcribed into RNA to create the RNA pool used for the next rounds of selection. Selection rounds were repeated 6 to 10 times with increased selection pressure (by reducing the amount of input RNA). Illumina next generation sequencing of human T-cell exosomes was performed on the enriched sequences. To obtain the motifs shown in FIG. 4, raw sequencing files were processed and trimmed of Illumina adapters and the reads per million (RPM) of each sequence was quantified and ranked. Sequences that were enriched were then analyzed further to generate conserved RNA motifs in the software MEME suite. The top 100 sequences at the end of round seven were those that were further analyzed. These sequences were consistently enriched across all sequencing rounds as analyzed using a specialized comparison algorithm. Conserved motifs were generated from sequences that were highly enriched into T cell exosomes. Each sequence motif comprises stacks of nucleotide symbols, with one stack representing each position in the sequence, as illustrated in the Discovered Motif column of FIG. 4. The overall height of the stack indicates sequence conservation at that position, while the height of nucleotides within the stack indicates the relative frequency of the respective nucleotide at that position. Each of these sequences are statistically significant based on their E-values, as shown in FIG. 4. 70% of the enriched RNA sequences in the top 20 most enriched sequences contained either full or partial (>75%) of the discovered motifs.

As will be recognized by those skilled in the art, exosomes are membrane-bound vesicles secreted by cells. They belong to the group of extracellular vesicles that collectively include exosomes, microvesicles, apoptotic bodies, and other extra-vesicular populations. Their size is in the range of 30-150 nanometers. Enriched proteins in exosomes include but are not limited to endosomal and transmembrane markers such as tetraspanins (CD63, CD9, CD81), integrins, cell adhesion molecules (EpCAM), growth factor receptors, heterotrimeric G proteins, and phosphatidylserine-binding MFG-E8/lactadherin. Further, exosomes may be enriched in endosome or membrane-binding proteins (Tsg101), annexins, Rabs, and signal transduction or scaffold proteins. However, the presence of any such protein constituents is not required for a released vesicle to be considered an exosome. In particular, exosome membrane composition is varied and may comprise different proportions of phospholipids (such as phosphatidylinositol, phosphatidylethanolamine, phosphatidylcholine, and phosphatidylserine) as well as their “-lyso” and “glycerol” derivatives, sphingomyelin, glycosphingolipids, ceramides, cholesterol (and esterified cholesterol), as well as lysobisphosphatidic acid (LBPA) among other lipids. The exosomes used to illustrate embodiments of the disclosure that are modified using the EXO-Codes as further described herein can deliver the cargo to targeted cells. In certain approaches the disclosure comprises modulation of nucleic acid or cargo sorting to exosomes.

The disclosure includes comparing the effects of any modified polynucleotide and/or modified exosome and/or modified cellular composition to a suitable reference. The reference can comprise any suitable control, value or measurement of the function of the modified polynucleotides and/or modified exosomes and/or modified cellular compositions, such as a standardized curve, a titration, the area under a curve, or a comparison to the capability of naturally occurring compositions or processes, including but not limited to the efficiency, kinetics, amount, etc. of RNA-exosome sorting in unmodified or other control systems. In embodiments, a control comprises a different type of cell than a cell that preferentially enriches and secretes exosomes comprising an EXO-Code described herein, e.g., a cell that comprises a polynucleotide comprising an EXO-Code described herein, but wherein the cell is not a lymphocyte, such as a T cell. In embodiments, the control comprises a polynucleotide that does not comprise an EXO-Code described herein. Representative and non-limiting control polynucleotide sequences are described, for example, in the Figures of this disclosure. In embodiments, the control polynucleotide is an RNA polynucleotide comprising a randomized sequence.

In embodiments, any one of the RNA EXO-Code sequences of this disclosure can comprise or consist of the sequences or segments of the sequences presented this disclosure. In certain embodiments, the EXO-Code sequences comprise or consist of between 3-70 nucleotides. Exosomes with a diameter of 30-150 nm have an internal radius of approximately 10-70 nm, thus the internal volume of one exosome ( 4/3 πR³) is approximately 4×10⁻²⁴−1.5×10⁻²¹ m³. The volume of one average 50 kDa protein or 100 nt RNA molecule is approximately 6×10⁻²⁶ m³. Thus, each exosome is expected to be able to accommodate approximately 70-25 000 small RNA or protein molecules (see, for example, Li, et al. Analysis of the RNA content of the exosomes derived from blood serum and urine and its potential as biomarkers, Phil. Trans. R. Soc. B369: 20130502, dx.doi.org/10.1098/rstb.2013.). Therefore even very large mRNAs may be feasibly incorporated into exosomes, provided an active packaging apparatus.

In certain aspects the disclosure comprises an EXO-Code comprising reagent that includes an RNA segment, wherein the RNA segment comprises an EXO-Code sequence, and wherein the agent further comprises a cargo moiety. The cargo moiety can be selected from polynucleotides, peptides, polypeptides, proteins, fluorophores, and small (drug) molecules.

EXO-Code polynucleotides may comprise modifications to improve their function, bioavailability, stability, and the like. For example, EXO-Code containing polynucleotides may include modified nucleotides and/or modified nucleotide linkages. Polynucleotides comprising such modifications may be referred to herein for convenience as RNA polynucleotides. Suitable modifications and methods for making them are well known in the art. Some examples include but are not limited to polynucleotides which comprise modified ribonucleotides or deoxyribonucleotides. For example, modified ribonucleotides may comprise methylations and/or substitutions of the 2′ position of the ribose moiety with an —O-lower alkyl group containing 1-6 saturated or unsaturated carbon atoms, or with an —O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In embodiments modified nulceotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof.

In certain aspects, such as for modulating the expression of a gene in a target cell or modifying a property or function of an exosome, and/or for reprogramming exosomes, the EXO-Code containing polynucleotides may comprise a functional polynucleotide segment. For example, the EXO-Code containing polynucleotides may be adapted for use in RNA interference (RNAi) mediated silencing or downregulation of a target mRNA or may be adapted for delivery of microRNA (miRNA) or other non-coding RNA (ncRNA). This can be achieved for example by joining the EXO-Code sequence to at least one RNAi agent. RNAi agents may be expressed in cells as short hairpin RNAs (shRNA). shRNA is an RNA molecule that contains a sense strand, antisense strand, and a short loop sequence between the sense and antisense fragments. shRNA is exported into the cytoplasm where it is processed by dicer into short interfering RNA (siRNA). siRNA are 21-23 nucleotide double-stranded RNA molecules that are recognized by the RNA-induced silencing complex (RISC). Once incorporated into RISC, siRNA facilitate cleavage and degradation of targeted mRNA. Thus, for use in RNAi mediated silencing or downregulation of a target RNA, the polynucleotide component may be either siRNA, shRNA, or miRNA. Any RNA or mRNA can be targeted. In non-limiting embodiments, the well-known Snail or Slug mRNA, or the S100A4 mRNA, or a combination thereof is targeted.

The EXO-Code containing polynucleotides may or may not encode a protein, including but not necessarily limited to a protein that is intended to facilitate RNA and/or exosome localization and/or visualization, or a protein that is capable of exerting a function in an exosome and/or in a target cell. In certain aspects the EXO-Code containing polynucleotides may encode and/or be modified to be attached to a protein that produces a detectable signal, including but not necessarily limited to a visually detectable signal, a fluorescent signal, etc. In certain approaches the EXO-Code containing polynucleotides may be covalently linked to any peptide or polypeptide. The type, sequence and function of such moieties are not particularly limited. In certain approaches the EXO-Code containing polynucleotide can be linked to a functional protein or fragment thereof. In certain embodiments the protein is selected from enzymes, receptor ligands, transcriptional factors, growth factors, antibodies or antigen-binding fragments thereof, peptide or protein immunogens that can be used for stimulating an immune response (i.e., a vaccine), protein-based chemotherapeutic agents, and toxins. In certain embodiments, the linked protein comprises insulin, a growth hormone or a growth hormone releasing factor, a platelet derived growth factor, an epidermal growth factor, any insulin-like growth factor, a clotting factor, an interferon, any interleukin, a lymphotoxin, and the like. In embodiments the linked protein comprises a protein-based toxin, such as enzymatically active toxins which include but are not limited to diphtheria A chain, nonbinding active fragments of diphtheria toxin, exotoxin A chain (from Pseudomonas aeruginosa), ricin A chain, abrin A chain, modeccin A chain, alpha sarcin, Aleurites fordii proteins, dianthin proteins, and Phytolaca americana proteins (PAPI, PAPII, and PAP-S. In general, protein with a volume of 1.5×10⁻²¹ m³ would be expected to be able to be present inside exosomes with a diameter of 30-150 nm. In particular, exosomes with a diameter of 30-150 nm have an internal radius of approximately 10-70 nm, thus the internal volume of one exosome ( 4/3 πR³) is approximately 4×10⁻²⁴−1.5×10⁻²¹ m³. The volume of one average 50 kDa protein or 100 nt RNA molecule is approximately 6×10⁻²⁶ m³. In non-limiting embodiments each exosome can accommodate any proteins with a volume smaller than 1.5×10⁻²¹ m³. In cases where the EXO-Codes mediate sorting to larger extracellular vesicles, larger proteins can be accommodated.

In embodiments, the exosomes may contain one or more components of a RNA-guided nucleases system, including but not limited to a clustered regularly interspaced short palindromic repeats (CRISPR) guide RNA. In certain embodiments, an exosome of this disclosure may also contain and/or encode one or more RNA-guided nucleases, including but not necessarily limited to a CRISPR associated enzyme (e.g. a Cas enzyme). In embodiments, the Cas is selected from a Class 1 or Class 2 Cas enzyme. In embodiments, a Type II or a Type V CRISPR Cas is used. In specific and non-limiting embodiments, the Cas comprises a Cas9, such as Streptococcus pyogenes (SpCas9). Derivatives of Cas9 are known in the art and may also be included in the exosomes. In a non-limiting embodiments, the Cas enzyme may be Cas12a, also known as Cpf1, or SpCas9-HF1, or HypaCas9.

In embodiments the EXO-Code sequence does not include the sequence 5′ UAG GGA AGA GAA GGA CAU AUG AU (SEQ ID NO:1) and/or does not include the sequence 5′ UU GAC UAG UAC AUG ACC ACU UGA 3′ (SEQ ID NO:2). In certain embodiments the EXO-Code sequence does not include the sequence: ACCCUGCCGCCUGGACUCCGCCUGU (SEQ ID NO:3). In certain embodiments the EXO-Code sequence does not include a GGAG motif. In embodiments, the EXO-Code sequence is not a component of an mRNA. In embodiments, the EXO-code sequence is not encoded by the genome of any organism.

In order for EXO-Code containing polynucleotides to exert their function they are introduced into exosomes using any of a variety of approaches. In embodiments EXO-Code containing polynucleotides are introduced into exosomes via cellular processing. Thus, EXO-Code containing polynucleotides can be introduced into cells which incorporate the EXO-Code containing polynucleotides into exosomes in the native cellular environment and exosome processing machinery, or they are introduced into an individual, such that they enter cells into the individual and are subsequently incorporated into exosomes. The EXO-Code containing polynucleotides are introduced into cells by using any suitable technique, examples of which include but are not limited to electroporation, incubation, cell activation, and transfection, lipid transfection, lipid delivery, liposomal delivery, polymer transfection, polymeric delivery, through peptide delivery (i.e. but not limited to cationic peptides, amphiphilic peptides, cell penetrating peptides), calcium or magnesium precipitation, and ion precipitation (also known as DNA-calcium phosphate precipitation). In embodiments, one or more EXO-Codes may be administered as a component of an RNA-protein complex, e.g., an RNP.

The disclosure thus includes in vitro cell cultures comprising one or more EXO-Code containing polynucleotides and one or more reagents that facilitate entry of the EXO-Code containing polynucleotides into the cells. The disclosure includes transcription templates encoding the EXO-Code containing polynucleotides, such as expression vectors configured to express the EXO-Code containing polynucleotides. Polynucleotides comprising EXO-Codes can be introduced directly into exosomes or other vesicular structures as described herein by using any suitable techniques, examples of which include but are not limited to electroporation, incubation, cell activation, and transfection, lipid transfection, lipid delivery, liposomal delivery, polymer transfection, polymeric delivery, through peptide delivery (i.e. but not limited to cationic peptides, amphiphilic peptides, cell penetrating peptides), calcium or magnesium precipitation, and ion precipitation (also known as DNA-calcium phosphate precipitation).

The disclosure includes compositions, including but not limited to pharmaceutical compositions suitable for human and/or veterinary uses, wherein the compositions comprise EXO-Code containing polynucleotides. Pharmaceutical compositions generally comprise at least one pharmaceutically acceptable excipient, carrier, diluent, and the like. The disclosure includes cell cultures modified to produce exosomes that contain the EXO-Code containing polynucleotides, cell culture medium comprising the exosomes, as well as isolated and/or purified exosome populations, wherein at least some of the exosomes in the population comprise EXO-Code containing polynucleotides. The disclosure includes pharmaceutical compositions comprising exosomes that contain EXO-Code containing polynucleotides. The disclosure includes methods of making the EXO-Code containing polynucleotides, such as by chemical synthesis, or in vitro or in vivo transcription. Also included are combinations of distinct polynucleotides comprising EXO-Codes wherein the combination has a greater than additive or synergistic effect on at least one EXO-Code/exosome sorting property and/or effect on a cell into which a modified exosome of this disclosure is introduced. Polynucleotides of this disclosure can comprise one, or more than one EXO-Code sequence, and can comprise more than one of the same EXO-Code sequence, or distinct EXO-Code sequences. In more detail, multivalency is a well-established approach in engineering higher affinity interactions between two moieties. An essentially unlimited number of EXO-Codes and combinations of distinct EXO-Codes could be incorporated into any single polynucleotide for the potential to achieve enhanced exosomal delivery. However, there may be an inverse relationship between increased affinity and size of the polynucleotide as more EXO-Codes are incorporated. Thus, in non-limiting embodiments, polynucleotides of this disclosure comprise 1, 2, 3, or more EXO-Codes. Further, although an advantage of EXO-Codes is realized when electroporated directly into living cells for active exosomal packaging, in applications where this is infeasible, direct exosome loading may be used. For the purposes of in vivo delivery the EXO-Codes could be loaded directly into isolated patient-derived exosomes via well-established electroporation protocols. They may also be incorporated into any synthetic lipidic delivery vehicle such as liposomes, cationic lipoplexes, other polymeric delivery vehicles or any host of delivery vehicle capable of delivering the EXO-Codes to the cytoplasm of recipient cells. These cells could then package the EXO-Codes into exosomes for in vivo exosome programming, or for other purposes.

Polynucleotides comprising the EXO-Codes may be introduced into an individual or cells using any suitable technique method and approach. Polynucleotides comprising the EXO-Codes may be introduced as modified or unmodified RNA, or by using for example a recombinant viral vector that can express the polynucleotides, including but not necessarily limited to lentiviral vectors, adenovirus vectors, and adeno associated viral vectors.

In embodiments, the disclosure includes obtaining T cells from an individual, modifying the cells ex vivo as described herein by introducing the polynucleotides comprising or encoding the EXO-Codes, and reintroducing the cells or their progeny into the individual for prophylaxis and/or therapy of a condition, disease or disorder, including but not necessarily limited to cancer. In embodiments, the cells modified ex vivo as described herein are used autologously. T cells modified according to this disclosure may have any HLA type.

In certain approaches polynucleotides comprising the EXO-Codes are introduced to an individual as a component of one or more cells, which may be autologous cells or heterologous cells, including but not necessarily limited to T cells, or cells that will differentiate into T cells. Thus, in embodiments, the disclosure includes introducing polynucleotides comprising the EXO-Codes described herein to hematopoietic stem cells that may be coaxed into differentiating into T cells. T cells used in this disclosure can be T helper cells, cytotoxic T cells, memory T cells, suppressor T cells, natural kill T cells, or gamma delta T cells. In general T cells are CD3+ cells that can be further distinguished from each other by subtype markers, such as CD4+ (T helper cells) and CD8+ (cytotoxic T cells). In certain embodiments the cells are thus CD4+ T cells, or CD8+ T cells, or so-called double positive T cells that are both CD4+ and CD8+ T Cells. In certain embodiments, T helper cells are T_{H}1, T_{H}2, T_{H}3, T_{H}17, T_{H}9, or T_{FH} cells. Memory T cells can be either CD4+ or CD8+ and typically express CD45RO. Memory T cells can be central memory, effector memory, or tissue resident T cells. In non-limiting examples, regulatory T cells, such as CD4+ T_{reg} cells, may be FOXP3+ T_{reg} cells or FOXP3-T_{reg} cells. In embodiments, the cells comprising EXO-Codes described herein may comprise T cells that express a Bi-specific T-cell engager (BiTE), a bispecific killer cell engager (BiKE), or a chimeric antigen receptor (CAR) (e.g. CAR T cells). In embodiments, the T cells express an engineered T cell receptor, and thus may express an engineered an alpha and beta chain T cell receptor. In embodiments, and EXO-Code containing polynucleotide is used to reprogram exosomes secreted by T cells, or other lymphocytes, or their precursors.

In certain embodiments, EXO-Code containing polynucleotides are used in connection with lymphocytes that are involved in promoting tolerance to one or more antigens. Among the T cell types responsible for peripheral tolerance and immune suppression, regulatory T cells (Tregs) are believed to be important. Naturally occurring regulatory T cells represent 5-10% of total CD4+ T cells and can be defined based on expression of CD25 and FOXP3. Accordingly, in certain implementations the disclosure is related to inducing, promoting, enhancing, or cooperating with immune tolerance in individuals who have autoimmune disorders. In embodiments, the method can accordingly be adapted for use with tolerogenic agents, including but not limited to inhibitors of the mammalian target of rapamycin (mTOR). In embodiments, the mTOR inhibitor is rapamycin, or a rapalog. In embodiments, the mTOR inhibitor comprises Sirolimus, Temsirolimus, Everolimus, Deforolimus, or a second generation mTOR inhibitor generally known to function as ATP-competitive mTOR kinase inhibitors, and/or mTORC1/mTORC2dual inhibitors. In embodiments, the tolerogenic agent comprises a cytokine or a chemokine or a growth factor or an interferon or a transcription factor, or other small molecule drugs that may include but are not limited to retinoic acid or mycophenolic acid. In embodiments a combination of tolerogenic agents can be used. In embodiments, a method involves introducing EXO-Code containing polynucleotides into T cells from, in, or to be introduced to an individual diagnosed with, suspected of having, or at risk for developing or relapsing, wherein the autoimmune disease is Addison's disease, Alopecia areata, Celiac disease, Chagas disease, Congenital heart block, Crohn's disease, Dermatitis herpetiformis, Dermatomyositis, Endometriosis, Fibromyalgia, Graves' disease, Guillain-Barre syndrome, Hashimoto's thyroiditis, Hypogammalglobulinemia, Juvenile arthritis, Juvenile diabetes (Type 1 diabetes), Lupus, Lyme disease, Meniere's disease, Multiple sclerosis, Myasthenia gravis, PANDAS, Peripheral neuropathy, Pernicious anemia, Primary biliary cirrhosis, Psoriasis, Psoriatic arthritis, Reactive Arthritis, Rheumatoid arthritis, Scleritis, Scleroderma, Sjogren's syndrome, Sperm & testicular autoimmunity, Type 1 diabetes, Ulcerative colitis, Vasculitis, or Vitiligo.

In certain approaches the disclosure thus comprises obtaining T cells from an individual who is diagnosed with or suspected of having cancer, modifying the T cells such as by introducing into them polynucleotides comprising the EXO-Codes described herein, and reintroducing the modified T cells into the individual. Without intending to be bound by any particular theory, it is considered that the T cells will preferentially enrich and secrete exosomes that contain the polynucleotides comprising the EXO-Codes, and as such any cargo that is joined to the polynucleotides will also be secreted in the exosomes. In this regard, it is considered that T cells that infiltrate tumors and/or directly recognize tumors may be particularly useful for combatting solid tumors because the exosomes will be secreted proximal to the tumor, thereby increasing the local concentration of the exosomes and their cargo. Thus, the disclosure in certain implementations is expected to be suitable for use with tumor infiltrating lymphocytes (or other T cells) in adoptive cell transfer therapy. Alternatively or additionally, T cells that secrete the exosomes into lymph, or that are locate themselves within tissues that are the location of a cancer may also have particular value. Mixtures of different types of T cells, and mixtures of T cells with other cell types, are included in this disclosure. In another embodiment, T cells from a patient may be obtained using any suitable technique, cultured if desired, and exosomes isolated from the T cells may be modified by introducing into them EXO-Code containing polynucleotides as described herein.

It should be recognized however, the present disclosure is not limited to treating any particular cancer type or tumor. Thus, cancers treated according to embodiments of this disclosure can be any type of cancer. In embodiments the cancer is a solid tumor which may be a solid tumor that is at risk for metastasis. In embodiments the individual may have a tumor that is at risk of or is undergoing metastasis. The individual may have previously had a metastatic tumor and is at risk for recurrence of a tumor and/or metastasis of it. In embodiments the cancer may be any one of fibrosarcoma, myxosarcoma, liposarcoma, chondrosarcoma, osteogenic sarcoma, chordoma, angiosarcoma, endotheliosarcoma, lymphangiosarcoma, pseudomyxoma peritonei, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, head and neck cancer, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, thymoma, Waldenstrom's macroglobulinemia, and heavy chain disease.

Accordingly, in certain embodiments a composition comprising a polynucleotide comprising an EXO-Code or a polynucleotide encoding such EXO-Code is introduced to an individual in need thereof, wherein an effective amount of polynucleotide comprising an EXO-Code is administered to or expressed in the individual such that the expression of at least one cancer related RNA is inhibited or eliminated, or an expressed RNA, such as an mRNA, is degraded, or translation of an mRNA is inhibited. In embodiments such administration results in an improvement in one or more symptoms of a disease or disorder such as cancer in the individual.

The approaches of this disclosure can be combined with any other anti-cancer approach, including but not necessarily limited to being used with chemotherapeutic agents, biologic agents such as antibodies and derivatives thereof, checkpoint inhibitors, radiation, and surgical interventions.

Those skilled in the art will recognize how to separate modified exosomes made according to this disclosure. In general exosomes are collected from a cellular supernatant and can be isolated by differential centrifugation according to well-known protocols. Exosomes comprising polynucleotides can be separated from those that do not comprise polynucleotides and subpopulations of exosomes can be obtained. EXO-Code containing polynucleotides can be isolated from the exosomes using standard approaches. Sequences of the EXO-Codes can be determined using standard approaches including but not necessarily limited to making and amplifying cDNA and determining the cDNA sequence using known sequencing techniques and apparatus, including but not limited to high throughput approaches. The disclosure thus includes methods of making, screening, and identifying EXO-Code sequences as described further herein. In general, methods for identifying EXO codes comprise subjecting a plurality of EXO-Codes having distinct sequences to a native intracellular environment which harnesses the endogenous cellular trafficking machinery to sort the EXO-Codes to exosomes. This approach is termed POSTAL: Procedure for Organelle-Specific Targeting by Aptamer Libraries. By chemically synthesizing RNA sequences of high diversity, we introduce sequences that have the potential to outperform natural miRNA and mRNA in their ability to sort to exosomes. Thus, this method has the potential to select for sequences that show improved exosomal enrichment in T cells when compared to existing approaches that look for motifs only within endogenous miRNAs. In certain embodiments EXO-Code containing polynucleotides of this disclosure show preferential enrichment in exosomes relative to naturally occurring RNA polynucleotides that are endogenously sorted to exosomes. In certain embodiments EXO-Code containing polynucleotides of this disclosure exhibit at least 2-1000 fold enrichment, inclusive, and including all integers and ranges of integers there between, by T cells. The enrichment in exosomes may be determined by comparison to a value obtained from determining enrichment using a wild type or endogenously occurring sequence, or a randomized sequence.

The disclosure includes kits for making, screening, and using EXO-Codes and polynucleotides comprising them for a wide variety of research, therapeutic and prophylactic approaches that will be apparent to those skilled in the art given the benefit of the present disclosure. The kits may comprise reagents for making and screening exosomes and may include EXO-Code containing polynucleotides and may contain reagents useful for introducing such polynucleotides into cells.

In one aspect the disclosure includes a plurality, such as a library. that includes at least two distinct polynucleotides which comprise a distinct EXO-Code sequences that are described herein. Thus the library may contain between 2 and 1000 distinct EXO-Code sequences. Accordingly, the disclosure includes a library comprising between 2 and 10000 distinct EXO-Code sequences wherein at least one of the EXO-Code sequences is described herein. In embodiments, an EXO-Code sequence is selected from the library, and T cells are modified to include and/or express a polynucleotide that contains the EXO-Code.

In one approach the disclosure comprises selecting a cell or cell type for modification by contacting it with a modified exosome of this disclosure. The method generally comprises mixing a cellular composition, such as T cells, with the modified exosomes such that the exosomes are taken up by the cells and the modified polynucleotide that comprises the EXO-Code and any particular cargo with which the polynucleotide has been modified is released into the cytoplasm. Subsequently the modified polynucleotide that comprises the EXO-Code may exert its effect in the target cell. These approaches can be used in vitro and are expected to be suitable for use in vivo. Thus, the disclosure includes selecting an individual in need of modification and/or reprogramming of exosomes, and administering to the individual an EXO-Code containing polynucleotide that is encompassed herein.

The following tables provide representative and non-limiting examples of EXO-Codes identified according to the foregoing description.

TABLE 1
RNA sequences identified after selection round
3. Top 50 sequences are shown.

| | | Reads | RPM | SEQ ID |
|---|---|---|---|---|
| Sequence | Rank (x) | (x) | (x) | NO: |
| GCAGUGAAAGGGACGGUUGC | 1 | 56 | 12.98 | 4 |
| UGUGUGUCCCCACAGCAGUG | 2 | 42 | 9.74 | 5 |
| CACUCGCGAGUCGCGGCAGG | 3 | 38 | 8.81 | 6 |
| UGAUGUAUUGGUAAGUUUCG | 4 | 36 | 8.35 | 7 |
| UUUCGUGUUUAGCGUACACA | 5 | 35 | 8.11 | 8 |
| GGUCCAAGCCGAGUCGUGCG | 6 | 27 | 6.26 | 9 |
| CUGGGAAUUGGCGUGCGUGA | 7 | 27 | 6.26 | 10 |
| AAGUUAAUAUCACACAUCUC | 8 | 26 | 6.03 | 11 |
| GCUUCAUCAUCUCGGGGCAC | 9 | 26 | 6.03 | 12 |
| UAGCAGAGCACUUCCUCACA | 10 | 25 | 5.8 | 13 |
| GAGGCCUGUGCUAGUAGUGA | 11 | 25 | 5.8 | 14 |
| CCGGACAAGUGCGGUUUGUC | 12 | 25 | 5.8 | 15 |
| ACAAUCUCCGGCCCACGAGA | 13 | 24 | 5.56 | 16 |
| GCGGGUUAUGGAAGCCGGUC | 14 | 23 | 5.33 | 17 |
| GGGCAAUGCUGAAGCGUCGG | 15 | 22 | 5.1 | 18 |
| GUUGUACAUGCACACAUCUC | 16 | 21 | 4.87 | 19 |
| ACAAUCUCCGACCCACGAGA | 17 | 21 | 4.87 | 20 |
| AGUGAGUAGCAGAUGCUAUG | 18 | 21 | 4.87 | 21 |
| CAUCAAGUUACGCCCGCGCA | 19 | 21 | 4.87 | 22 |
| GCGCGGUCUCGUUGGCAUAG | 20 | 21 | 4.87 | 23 |
| GUAUUGAUUAAAAGCCACAC | 21 | 20 | 4.64 | 24 |
| CUAGGAUGGGUACUGCCUGC | 22 | 20 | 4.64 | 25 |
| GCGAAUUGACGUGGUUGGCG | 23 | 20 | 4.64 | 26 |
| AGCUUGGCAAGCUGCCGCUG | 24 | 20 | 4.64 | 27 |
| GGGAGGAAAUGUGAGCGUGG | 25 | 19 | 4.41 | 28 |
| AAGUAUUUCUUUCCACACAU | 26 | 19 | 4.41 | 29 |
| AGCGGACAAAGGCAGAGGGG | 27 | 19 | 4.41 | 30 |
| ACGUGUGGCUGAGGGGCAGC | 28 | 19 | 4.41 | 31 |
| GAUGCACGCGUGUUCGGCCG | 29 | 18 | 4.17 | 32 |
| GGGUAUGGCGGACUAUGGCC | 30 | 18 | 4.17 | 33 |
| GCGUGGACGAGGGGUAUGUG | 31 | 18 | 4.17 | 34 |
| GCGGUUGGCACGGGGCCUGG | 32 | 18 | 4.17 | 35 |
| GGUCGGGUCGGUUGCACGGG | 33 | 18 | 4.17 | 36 |
| GCUAGGUGCUGCCACUGUUC | 34 | 17 | 3.94 | 37 |
| GUCGGCCAGGGACGCGUGCG | 35 | 17 | 3.94 | 38 |
| UUUCAUGUAUUGGUGUGGCA | 36 | 17 | 3.94 | 39 |
| CCGUCGUUAGCCAAUUGCUC | 37 | 17 | 3.94 | 40 |
| CGGGAGCUGGCUGGGCAGUG | 38 | 16 | 3.71 | 41 |
| GACGUUGGGGGUGAGUACAG | 39 | 16 | 3.71 | 42 |
| GUGCUGCACGCUGGGGAGUG | 40 | 16 | 3.71 | 43 |
| GCGUGGCUUGCCGUGGUUAC | 41 | 16 | 3.71 | 44 |
| GUGUCGCAAGGCCCUUGCAG | 42 | 16 | 3.71 | 45 |
| CUGGUAAAAUGGCUGCCGGA | 43 | 16 | 3.71 | 46 |
| GCACCAUUGUAAGGCGGACG | 44 | 16 | 3.71 | 47 |
| GGGGCGAGGGAGGUCGUUUG | 45 | 16 | 3.71 | 48 |
| GAGGUUUAGGUUGGCGGGCG | 46 | 16 | 3.71 | 49 |
| AGCACCAGGGGUGGCGGAGG | 47 | 16 | 3.71 | 50 |
| CGGAGUUAGGAUGCGCCGCG | 48 | 16 | 3.71 | 51 |
| AGGCAACGGGGCGGGAGGGC | 49 | 16 | 3.71 | 52 |
| UGGAGAACUGGUGUGCUGGA | 50 | 16 | 3.71 | 53 |

TABLE 2
RNA sequences identified after selection round
5. Top 50 sequences are shown

| | | | | SEQ |
|---|---|---|---|---|
| | | Reads | | ID |
| Sequence | Rank (x) | (x) | RPM (x) | NO: |
| AAGGCCGGUGCUAGUAGUGA | 1 | 3975 | 898.66 | 54 |
| ACACAUCCCGAGCCCACGAG | 2 | 184 | 41.6 | 55 |
| UUUCGUGUUUAGCGUACACA | 3 | 135 | 30.52 | 56 |
| AGUACAUGACCACACACAUC | 4 | 108 | 24.42 | 57 |
| GAGGCCUGUGCUAGUAGUGA | 5 | 89 | 20.12 | 58 |
| CUAGUACAUGACCACACACA | 6 | 86 | 19.44 | 59 |
| UGAUGUAUUGGUAAGUUUCG | 7 | 79 | 17.86 | 60 |
| UGUGUGUCCCCACAGCAGUG | 8 | 74 | 16.73 | 61 |
| GAGGCCGGUGCUAGUAGUGA | 9 | 70 | 15.83 | 62 |
| ACACACUCCGGCCCACGAGA | 10 | 69 | 15.6 | 63 |
| UCAUUGUGCUGAAACACAUC | 11 | 66 | 14.92 | 64 |
| GGGGGAUGGAUGGAGGGGCG | 12 | 54 | 12.21 | 65 |
| ACACAUCCCCGAGCCCACGA | 13 | 50 | 11.3 | 66 |
| AGUACAUGACCACUUGAACA | 14 | 50 | 11.3 | 67 |
| CCGUGGCGUGUUGGACACAU | 15 | 49 | 11.08 | 68 |
| UAGCAGAGCACUUCCUCACA | 16 | 43 | 9.72 | 69 |
| CCACCUUUGCCGGAUGCCCG | 17 | 42 | 9.5 | 70 |
| GGGGGGCUGGGUGUCCGUGG | 18 | 40 | 9.04 | 71 |
| GGAGGGAAGGAGGGUGCCGG | 19 | 40 | 9.04 | 72 |
| UGAUAGUGGAGAGCGCGCGG | 20 | 39 | 8.82 | 73 |
| GGGCGAAAUUGGCAUGGCCG | 21 | 39 | 8.82 | 74 |
| GGAGGGGGGAGGGGGCGGUG | 22 | 38 | 8.59 | 75 |
| AGAGGGAGGGGGUGUCGUGG | 23 | 38 | 8.59 | 76 |
| UGAUGUAUUUGGUUUGCAAG | 24 | 37 | 8.36 | 77 |
| GAAUGCUUGUUCAGACACAU | 25 | 37 | 8.36 | 78 |
| CUAGUACAUGACCACUUGAA | 26 | 36 | 8.14 | 79 |
| AAGGCCGGCGCUAGUAGUGA | 27 | 35 | 7.91 | 80 |
| GGUGCGGCUGUAGUUCCGGG | 28 | 35 | 7.91 | 81 |
| CAUGACCACUUGAACACAUC | 29 | 35 | 7.91 | 82 |
| GGAGGGAGGAGGGGCGCGGG | 30 | 34 | 7.69 | 83 |
| AGGGGGAGGAGGCGGGAUGG | 31 | 34 | 7.69 | 84 |
| GGAGGCGGUGAGGGUUGUGG | 32 | 34 | 7.69 | 85 |
| GCGCGAUAUGGAGGGACUGC | 33 | 34 | 7.69 | 86 |
| GGGAGGAGGGUGGCGCGCGG | 34 | 33 | 7.46 | 87 |
| CCUCGGACAUGUCUUGGUGC | 35 | 33 | 7.46 | 88 |
| UAGGCUUUGAAACAGAUUGC | 36 | 33 | 7.46 | 89 |
| UAGCGGGGAGGGAGGCGCGG | 37 | 32 | 7.23 | 90 |
| CGAGGGUGUGUCCUGUGGGCU | 38 | 32 | 7.23 | 91 |
| GUGUGGGGCGAGGCGGUGGG | 39 | 32 | 7.23 | 92 |
| GUAUUGAUUAAAAGCCACAC | 40 | 32 | 7.23 | 93 |
| GGUUAAUUUUAUGUGUCAAC | 41 | 32 | 7.23 | 94 |
| UGGAUGGCAGUCGUCACGUG | 42 | 31 | 7.01 | 95 |
| GUUGGGGAUGUCUGUGUGGG | 43 | 31 | 7.01 | 96 |
| CGAUUCUGGCGCGAUCCUGG | 44 | 31 | 7.01 | 97 |
| GGGGAGGACGGGGGGCGUGG | 45 | 30 | 6.78 | 98 |
| GGGAGGGGGGUGGUCCGUUG | 46 | 30 | 6.78 | 99 |
| UGCGAUGUUGUGAGUGGCCC | 47 | 30 | 6.78 | 100 |
| GUGCGUAUGUUGUGUGGGGG | 48 | 30 | 6.78 | 101 |
| CCGGCGGGGUUUGGGGCCUG | 49 | 30 | 6.78 | 102 |
| GAUGUCUUAUCGUCAUGUGU | 50 | 30 | 6.78 | 103 |

TABLE 3
RNA sequences identified after selection round
7. Top 50 sequences are shown

| | Rank | Reads | | SEQ ID |
|---|---|---|---|---|
| Sequence | (y) | (y) | RPM (y) | NO: |
| UGAUGUAUUUGGUUUGCAAG | 1 | 617 | 113.43 | 104 |
| UGAUGUAUUGGUGGGUUUCG | 2 | 383 | 70.41 | 105 |
| UUUCGUGUAUCCUAGUUGCU | 3 | 279 | 51.29 | 106 |
| UUAUUGCAUCUGUAGUAGUU | 4 | 262 | 48.17 | 107 |
| UGAAAUGAGACUGGUUUUGC | 5 | 247 | 45.41 | 108 |
| UGAAUUGUACAGAAGCUUGA | 6 | 213 | 39.16 | 109 |
| UUGAAAUGUGCUGUUGCAGA | 7 | 211 | 38.79 | 110 |
| UUGAUGUACGUGUAAGUUUA | 8 | 202 | 37.14 | ill |
| UGCUUGUACAUUGUUUACUU | 9 | 188 | 34.56 | 112 |
| GUAUUUGUUUUGAUUGCUGC | 10 | 183 | 33.64 | 113 |
| UACACAUGAACAUAGUGACA | 11 | 169 | 31.07 | 114 |
| AUGCAUUAGUUACUCGAAUG | 12 | 160 | 29.42 | 115 |
| AUUUUCGUGUACGAAUGGUU | 13 | 142 | 26.11 | 116 |
| GAUGUGCGAGUUUUAUAUUG | 14 | 127 | 23.35 | 117 |
| UCAUGGUAACUAACUUGUUG | 15 | 124 | 22.8 | 118 |
| AUGGUAGUAGCAAUUGUAAA | 16 | 123 | 22.61 | 119 |
| UAAUGUAGCAAAGUUUUUUA | 17 | 121 | 22.25 | 120 |
| GGGGAGGAGGGAGCGCGCGG | 18 | 119 | 21.88 | 121 |
| CCAGGGAGGACGGGUCGUGG | 19 | 118 | 21.69 | 122 |
| UUGACGUACGGUUAUCUAUA | 20 | 117 | 21.51 | 123 |
| GGAGGGAGGAGGGGCGCGGG | 21 | 115 | 21.14 | 124 |
| ACAUGUGAUUGGUUGCAGUG | 22 | 114 | 20.96 | 125 |
| GGAGGGAGAGGAGGGCGCGG | 23 | 112 | 20.59 | 126 |
| GUGCUAUGGAAUUAUAUUGA | 24 | 112 | 20.59 | 127 |
| GGGGAGGACGGGGGGCGUGG | 25 | 111 | 20.41 | 128 |
| AAUUACACUGUGCUAGGAUG | 26 | 111 | 20.41 | 129 |
| GCAUUUAUGACUAAGUCUUG | 27 | 111 | 20.41 | 130 |
| GCUGGGGGAGGGGCGUUGGG | 28 | 110 | 20.22 | 131 |
| GCGGGAAGGGGGGCCUGUGG | 29 | 109 | 20.04 | 132 |
| GAGGUUGGGGAGGGCCGUGG | 30 | 107 | 19.67 | 133 |
| GGGGGAUGGAUGGAGGGGCG | 31 | 106 | 19.49 | 134 |
| GGAGGGGGGAGGGGGCGCGG | 32 | 105 | 19.3 | 135 |
| GGGGCUGGGGCGUGGUGUGG | 33 | 105 | 19.3 | 136 |
| GGGUAGGUGGAGGGCGUUGG | 34 | 104 | 19.12 | 137 |
| ACAGACGGUUGCUUGCGGGG | 35 | 99 | 18.2 | 138 |
| GGGGGAAGCGUGUUCGGUGG | 36 | 99 | 18.2 | 139 |
| GGGGGGAGGAGGGGGCUGCG | 37 | 98 | 18.02 | 140 |
| GCAUGUAUUGGUUUUUGGUU | 38 | 98 | 18.02 | 141 |
| UGAAGCUGUACAAAGUUUGC | 39 | 96 | 17.65 | 142 |
| UAGCAGAACGGCGCGUGUGG | 40 | 95 | 17.47 | 143 |
| GGGCGCACAUAUGUUGGUGG | 41 | 94 | 17.28 | 144 |
| GGGGGACGGGCGGGGGUUGG | 42 | 94 | 17.28 | 145 |
| UCAUUGUGCUGAAACAAUCU | 43 | 94 | 17.28 | 146 |
| GGGGAAGGAGGCGGGCGUGG | 44 | 93 | 17.1 | 147 |
| GGGGAGGGAAGGCGGGCGGA | 45 | 93 | 17.1 | 148 |
| UUUGUGUACAAAGCAGAUUC | 46 | 93 | 17.1 | 149 |
| AGUGUAUUGCGAUCAGUUGA | 47 | 93 | 17.1 | 150 |
| GGGGGGUGAGGGGGGACCGG | 48 | 91 | 16.73 | 151 |
| CGCUGGUCUGCCUGUGUGCG | 49 | 91 | 16.73 | 152 |
| GCACAAUCUCCGAGCCCACG | 50 | 91 | 16.73 | 153 |

TABLE 4
RNA sequences identified after selection round
9. Top 100 sequences are shown.

| | | | | SEQ | |
|---|---|---|---|---|---|
| | Rank | Reads | | ID | |
| Sequence | (z) | (z) | RPM (z) | NO: | |
| UUUCGUGUUUAGCGUACACA | 1 | 77239 | 13304.25 | 154 | T4 |
| UGAUGUAUUGGUAAGUUUCG | 2 | 17106 | 2946.47 | 155 | |
| AACUGUAUUGGUUAUACACA | 3 | 6543 | 1127.02 | 156 | |
| UAUAGAUGUGCUAGUUUGCA | 4 | 5185 | 893.11 | 157 | |
| AACAAUCUCCGAGCCCACGA | 5 | 4822 | 830.58 | 158 | |
| UUUCGUGUUUGGCGUACACA | 6 | 3984 | 686.24 | 159 | |
| UCAUUGUGCUGAAACACAUC | 7 | 3760 | 647.65 | 160 | |
| UGAUGUAUUGGUAAGUUUUG | 8 | 3575 | 615.79 | 161 | |
| AACUGUAUUGGUUGUACACA | 9 | 3527 | 607.52 | 162 | |
| AAGGCCGGUGCUAGUAGUGA | 10 | 3509 | 604.42 | 163 | |
| UUUCGUGUUUAGCGACACAU | 11 | 2527 | 435.27 | 164 | |
| UUUCGUGUUUAGCGUACAAU | 12 | 2164 | 372.74 | 165 | |
| UUUCGUGUUUAGCUUACACA | 13 | 1965 | 338.47 | 166 | |
| ACGUGUAUUGCUAACACAUC | 14 | 1563 | 269.22 | 167 | |
| ACAUGUAUUGGUUUUUGGUU | 15 | 1507 | 259.58 | 168 | T3 |
| UGAUGUAUUGGUGAGUUUCG | 16 | 1284 | 221.17 | 169 | |
| UUUUGUGUACUUGCAUUUCA | 17 | 1059 | 182.41 | 170 | |
| GCGUGUAUUACUAACACAUC | 18 | 1003 | 172.76 | 171 | |
| UGAUGUAUUUGGUUUGCAAG | 19 | 956 | 164.67 | 172 | |
| UGAUGUAUUGGUAGGUUUCG | 20 | 890 | 153.3 | 173 | |
| CACACAAUCUCCGAGCCCAC | 21 | 772 | 132.98 | 174 | |
| AGCUGUAUUGGUUAUACACA | 22 | 717 | 123.5 | 175 | |
| ACAUGUGAUUAGUUGCAAUG | 23 | 706 | 121.61 | 176 | |
| UAUAGAUGUGCUGGUUUGCA | 24 | 660 | 113.68 | 177 | |
| AACACAUUCCGAGCCCACGA | 25 | 622 | 107.14 | 178 | |
| UGAAGCUGUACAAAGUUUGU | 26 | 564 | 97.15 | 179 | |
| ACAUGUGAUUGGUUGCAAUG | 27 | 557 | 95.94 | 180 | |
| AGCUGUAUUGGUUGUACACA | 28 | 553 | 95.25 | 181 | |
| GCGUGUAUUGCUAACACAUC | 29 | 537 | 92.5 | 182 | |
| UGAUGUAUUGGUGGGUUUCG | 30 | 517 | 89.05 | 183 | |
| CUGAUGUCUCUUAUACAGAC | 31 | 489 | 84.23 | 184 | |
| AACACUCUCCGAGCCCACGA | 32 | 479 | 82.51 | 185 | |
| UUUGCAAGUGUACAGUUGUU | 33 | 477 | 82.16 | 186 | |
| UGAUGUGUUAGUUUGAAUGU | 34 | 383 | 65.97 | 187 | |
| GGGGGGCUGGGUGUCCGUGG | 35 | 375 | 64.59 | 188 | |
| UUUCGUGUUUAGCGUACAUC | 36 | 340 | 58.56 | 189 | |
| UUUCGUGUUUAGCGUACACU | 37 | 332 | 57.19 | 190 | |
| AACACAUCCCGAGCCCACGA | 38 | 323 | 55.64 | 191 | |
| ACGUGUAUUACUAACAAUCU | 39 | 298 | 51.33 | 192 | |
| UGAAUGUAGCUUAGUACAAA | 40 | 292 | 50.3 | 193 | |
| GAAAUGUACAAUGAUCACAC | 41 | 276 | 47.54 | 194 | |
| GGGGAGGACGGGGGGCGUGG | 42 | 275 | 47.37 | 195 | |
| AACACAUCCGAGCCCACGAG | 43 | 274 | 47.2 | 196 | |
| AACAACUCCGAGCCCACGAG | 44 | 274 | 47.2 | 197 | |
| AACACACUCCGGCCCACGAG | 45 | 274 | 47.2 | 198 | |
| UGACUGUCUCUUAUACAGAC | 46 | 271 | 46.68 | 199 | |
| UUACAAUGCGCUAGUUUUUG | 47 | 271 | 46.68 | 200 | |
| AACACAUCCCCGAGCCCACG | 48 | 269 | 46.33 | 201 | |
| CUGCUGUCUCUUAUACAGAC | 49 | 264 | 45.47 | 202 | |
| ACGUGUAUUACUAACACACU | 50 | 264 | 45.47 | 203 | |
| UUGAAGUGUACAUUGUCGUA | 51 | 256 | 44.1 | 204 | |
| UUAUUGCAUCUGUAGUAGUU | 52 | 248 | 42.72 | 205 | |
| UAAUGUAUUGGCAUAACUAC | 53 | 246 | 42.37 | 206 | |
| GAUGUAUAGUUUUGAUGCAC | 54 | 237 | 40.82 | 207 | |
| GAGGGAGGAGGAGGGCGGCG | 55 | 232 | 39.96 | 208 | |
| AACACCUCUCCGAGCCCACG | 56 | 225 | 38.76 | 209 | |
| GACAAUCUCCGAGCCCACGA | 57 | 221 | 38.07 | 210 | |
| UGAUGUAUUGGUGAGUUUUG | 58 | 209 | 36 | 211 | |
| AACUGUAUUGGUUAUACAAU | 59 | 209 | 36 | 212 | |
| GGGGAAGGAGGCGGGCGUGG | 60 | 206 | 35.48 | 213 | |
| GUAUUGUAGUAAUUACUGUA | 61 | 203 | 34.97 | 214 | |
| UUUUGUGUUUAGCGUACACA | 62 | 203 | 34.97 | 215 | |
| UGAUUGAUACUGUGUAAUUA | 63 | 192 | 33.07 | 216 | |
| UGAUAGUAUUGGUCUAUUCA | 64 | 191 | 32.9 | 217 | |
| UUGAUGUACGUGUAAGUUUA | 65 | 186 | 32.04 | 218 | |
| UUUCGCGUUUAGCGUACACA | 66 | 186 | 32.04 | 219 | |
| AACAUAUCUCCGAGCCCACG | 67 | 178 | 30.66 | 220 | |
| AUAACAAACUGUGCUAGACA | 68 | 176 | 30.32 | 221 | |
| AACAAUCUCCGACCCACGAG | 69 | 172 | 29.63 | 222 | |
| UGAUGUAUUGGUAGGUUUUG | 70 | 172 | 29.63 | 223 | |
| GGGGGGAGGAGGGGGCUGCG | 71 | 169 | 29.11 | 224 | |
| CGCACAAUCUCCGAGCCCAC | 72 | 168 | 28.94 | 225 | |
| GGGCUGGGGGGGGGCCGUGG | 73 | 168 | 28.94 | 226 | |
| UUUCGUGUUUAGCAUACACA | 74 | 168 | 28.94 | 227 | |
| UUUCGUGUUUAUCGUACACA | 75 | 164 | 28.25 | 228 | |
| UUUCGUGCUUAGCGUACACA | 76 | 163 | 28.08 | 229 | |
| CCAGGGAGGACGGGUCGUGG | 77 | 162 | 27.9 | 230 | |
| CUAGCGACGGUGCGGGGGUG | 78 | 162 | 27.9 | 231 | |
| CUUCGUGUUUAGCGUACACA | 79 | 155 | 26.7 | 232 | |
| UCAUGGAUACUAUGCAUUGA | 80 | 155 | 26.7 | 233 | |
| AACAAUCUCCGGCCCACGAG | 81 | 141 | 24.29 | 234 | |
| ACGUGUAUUACUGACACAUC | 82 | 141 | 24.29 | 235 | |
| UGAUGUUACUGUUGUUUCGA | 83 | 141 | 24.29 | 236 | |
| UUUCGUGUUUAGCGCACACA | 84 | 140 | 24.11 | 237 | |
| UGAUGUAUUUGGUUUGCAGG | 85 | 140 | 24.11 | 238 | |
| UUUCGUGUUCAGCGUACACA | 86 | 139 | 23.94 | 239 | |
| GGGGAGGAUAUGGCCUGUGG | 87 | 137 | 23.6 | 240 | |
| CCGUGGGGAGGGGAGCUCGG | 88 | 136 | 23.43 | 241 | |
| AGUGUAUUGUGUCAUACUGA | 89 | 136 | 23.43 | 242 | |
| GGGCGGGAAUCGUGGUGCGG | 90 | 135 | 23.25 | 243 | |
| GGAGGGAGGAGGGGCGCGGG | 91 | 135 | 23.25 | 244 | T2 |
| UUAUGUAAUGGCGAUUUACA | 92 | 135 | 23.25 | 245 | |
| GAGGUUGGGGAGGGCCGUGG | 93 | 134 | 23.08 | 246 | |
| UAUCAUGGUACAGUUUUGGC | 94 | 133 | 22.91 | 247 | |
| GACUGUAUUGGUUAUACACA | 95 | 132 | 22.74 | 248 | |
| GCACUGGUUUGUAACACAUC | 96 | 131 | 22.56 | 249 | |
| GGAGGGGGGAGGGGGCGCGG | 97 | 131 | 22.56 | 250 | |
| UUUCGUGUUUAGCGUACAAC | 98 | 129 | 22.22 | 251 | |
| UGCUUGUACAUUGUUUACUU | 99 | 129 | 22.22 | 252 | |
| CCGUGGGUGGCUGGGUGUGG | 100 | 128 | 22.05 | 253 | |

For the RNA sequences in Table 4, selection rounds 3, 5, 7, and 9 were sequenced. Sequence with read counts 15 standard deviations above the mean for each round were pooled and analyzed using MEME suite. Identified motifs were up to 12 nucleotides in length and could contain any number of repetitions. There were three motifs showing statistical significance. The 20N sequences from round 9 were then interrogated and the highest ranked sequences containing the bioinformatic-determined motifs were synthesized as RNA. Experimentally verified sequences are T4 (rank 1), T3 (rank 15) and T2 (ranked 91). The sequences T4, T3 and T2 are identified in the right column of Table 4 and are depicted in FIGS. 1-3. Certain sequences in the sequence listing are duplicated, but assigned different sequence identifiers. For the purpose of this disclosure, the sequence identifiers for T2, T3 and

T4 are shown on FIGS. 1-3 as representative examples of EXO-Codes, and are

(SEQ ID NO: 91)
GGAGGGAGGAGGGGCGCGGG (“T2:);
(SEQ ID NO: 168)
ACAUGUAUUGGUUUUUGGUU (“T3”);
and
(SEQ ID NO: 154)
UUUCGUGUUUAGCGUACACA (“T4”).

While the invention has been described through specific embodiments, routine modifications will be apparent to those skilled in the art and such modifications are intended to be within the scope of the present invention.

1. An RNA polynucleotide, optionally comprising one or more modified nucleotides, the RNA polynucleotide comprising a sequence that facilitates preferential enrichment of membranous vesicles with the RNA polynucleotides within T cells, and secretion of the membranous vesicles by the T cells, relative to enrichment of membranous vesicles with a control RNA polynucleotide by the T cells.

2. The RNA polynucleotide of claim 1, wherein the RNA polynucleotide comprises a motif sequence that is GUACMYGACSAC (SEQ ID NO: 255), WSVUGURYURSU (SEQ ID NO: 258), GRGAAGGACRUM (SEQ ID NO: 261), or GUCACACAGUCC (SEQ ID NO: 264).

3. The RNA polynucleotide of claim 1, comprising a sequence selected from the sequences in Table 1, Table 2, Table 3, or Table 4.

4. The RNA polynucleotide of claim 3, comprising a sequence that is
(SEQ ID NO: 91)
GGAGGGAGGAGGGGCGCGGG (“T2:);
(SEQ ID NO: 168)
ACAUGUAUUGGUUUUUGGUU (“T3”);
or
(SEQ ID NO: 154
UUUCGUGUUUAGCGUACACA (“T4”).

5. The RNA polynucleotide of claim 1, wherein the membranous vesicles comprise exosomes.

6. Modified eukaryotic cells comprising an RNA polynucleotide of claim 1.

7. The modified eukaryotic cells of claim 6, wherein the modified eukaryotic cells comprise lymphocytes.

8. The modified eukaryotic cells of claim 7, wherein the lymphocytes comprise T cells.

9. A method comprising introducing into eukaryotic cells an RNA polynucleotide of claim 1, to thereby produce modified eukaryotic cells comprising the RNA polynucleotide.

10. The method of claim 9, wherein the modified eukaryotic cells comprise lymphocytes.

11. The method of claim 10, wherein the lymphocytes comprise T cells.

12. The method of claim 11, wherein the T cells are isolated from an individual.

13. The method of claim 12, further comprising introducing the T cells comprising the RNA polynucleotide into the individual from which the T cells were isolated.

14. An isolated RNA polynucleotide of claim 1.

15. An isolated plurality of membranous vesicles comprising an RNA polynucleotide of claim 1.

16. The isolated plurality of membranous vesicles of claim 15, wherein the membranous vesicles comprise exosomes.

17. A pharmaceutical formulation comprising a polynucleotide comprising a sequence of claim 1.

18. The pharmaceutical formulation of claim 17, wherein the polynucleotide is comprised by a membranous vesicle.

19. The pharmaceutical formulation of claim 18, wherein the membranous vesicle comprises exosomes.

20. An expression vector encoding the RNA polynucleotide of claim 1.

21. A cell culture in which cells in the cell culture secrete exosomes, wherein the exosomes comprise a polynucleotide of claim 1.

22. A method comprising separating exosomes secreted from the cells in the cell culture of claim 21.
